SEQUENCE LISTING (4 sequences)

SEQ ID NO: 1 - RNA, 59 nt, Artificial Sequence
  Antisense RNA corresponds to Vibrio Harveyi dam gene
cuuuuucaucuacugcucuaucuaucgaccaaaaauuaaggcugcggaauguaacauau

SEQ ID NO: 2 - RNA, 54 nt, Artificial Sequence
  Antisense RNA corresponds to green fluorescent protein (GFP) gene
uaauucaacaagaauugggacaacuccagugaaaaguucuucuccuuuacucau

SEQ ID NO: 3 - DNA, 840 bp, Vibrio Harveyi
atgaaaaagcaacgagcctttcttaagtgggcaggaggcaaatacggtctggttgaagacatccaacgtcatttaccaccggctcgaaagctagttgaaccctttgttggtgctggctcggtttttctaaataccgactatgaccactatctactggcggatattaaccccgacctgattaatctctataacttactaaaagagcgtcctgaagagtacatctcagaagcgaagcgctggtttgttgcagagaacaatcgcaaagaagcgtacttgaatattcgcgccgagtttaataaaacggatgacgtgatgtaccgctcgttggcgttcctatacatgaaccgctttggctttaatggcttatgtcgttataacaaaaaaggcggctttaatgtcccgtttggttcttacaaaaagccttatttcccagaagcggagctagaattctttgctgaaaaagccaagaaagcgacgttcgtatgtgaaggttacccagaaacgttcagtcgagcgcgtaaaggcagcgtggtttattgcgatccaccgtacgcaccgttgtcgaacacggcgaactttacctcttatgctggcaacggctttacgctggatgatcaagctgcattggctgatattgcagagaaagccgcaactgaacgtggtatccctgttctgatctcaaaccatgacacgacattaacgcgtcgcctttatcatggtgcggagcttaatgtcgtaaaagtgaagcgaaccatcagtcgtaatggcagtggtcgtaataaagttgacgagttgctggcgctatttcgtgcacctgacgcggacaaatctgactcttaa

SEQ ID NO: 4 - DNA, 159 bp, Aedes aegypti
cccttcacaaacctggagaaaacgtccgtgctgcaggaaacgcggatgtttaacgagaccccggtcaatgcccgcaagtgtacccacatcctgacgaagattctgtatttgatcaatcagggagaacaactgggttccagagaggccaccgaatgtttc

(19) United States
(12) Patent Application Publication
(10) Pub. No.: US 2020/0149048 A1
(43) Pub. Date: May 14, 2020

(54) Transbiotic Regulation of Bacterial Gene Expression

(71) Applicant: Pebble Labs USA Inc., Santa Fe, NM (US)

(72) Inventors: Richard Sayre, Los Alamos, NM (US); Tatiana Vinogradova-Shah, Los Alamos, NM (US); Elena Sineva, Los Alamos, NM (US); Pedro Costa-Nunes, Albuquerque, NM (US)

(21) Appl. No.: 16/616,090

(22) PCT Filed: May 22, 2018
(86) PCT No.: PCT/US18/33976

§ 371 Date: Nov. 22, 2019

Related U.S. Application Data
(60) Provisional application No. 62/509,272, filed on May 22, 2017.

(51) Int. Cl. C12N 15/113; C12N 15/70; C12N 9/10; A61K 35/742; A61K 35/74

(52) U.S. Cl. CPC C12N 15/1137; C12N 15/70; C12N 9/1007; C12N 2310/11; A61K 35/742; A61K 35/74; C12Y 201/01072

(57) ABSTRACT

The inventive technology may include novel systems for regulation of the expression of bacterial genes through the introduction of antisense RNA (asRNA) that may disrupt expression of targeted pathogenic genes and/or their products (mRNA, proteins). In some embodiments, the inventive technology may include novel genetically engineered donor bacterial strains that are configured to efficiently and continuously deliver asRNA polynucleotides to a recipient pathogen and downregulate expression or one or more essential genes.

This application claims the benefit of and priority to U.S. Provisional Application No. 62/509,272, filed May 22, 2017. The entire specification and figures of the above-referenced application are hereby incorporated, in their entirety by reference.

### SEQUENCE LISTING

The instant application contains a Sequence Listing which has been submitted electronically in ASCII format and is hereby incorporated by reference in its entirety. Said ASCII copy, created on May 22, 2018, is named PCT6_Seq-Listing.txt and is 2 Kbytes in size.

### TECHNICAL FIELD

Generally, the inventive technology relates to novel transbiotic strategies for controlling disease-causing agents, including multi-drug resistant bacteria in a host eukaryotic organism. In particular, the inventive technology may include novel systems for regulating expression of bacterial gene expression through the introduction of antisense RNA (asRNA) that may disrupt expression of targeted pathogenic genes and/or their products (RNA, proteins). In some embodiments, the inventive technology may include novel genetically engineered donor bacterial strains configured to efficiently and continuously deliver asRNA polynucleotides to a recipient pathogen and downregulate expression of one or more essential genes in a host.

### BACKGROUND OF THE INVENTION

According to a report of World Health Organization (WHO), more than 20% of total deaths in the world are due to infectious diseases. The mechanisms of bacterial-mediated host infection are based on the interactions between the proteins of the pathogen and its host. Bacterial plant pathogens result in crop losses estimated to be in the hundreds of billions of dollars annually and have been directly responsible for increasing food insecurity worldwide.

Traditionally, pathogenic bacteria have been managed through the use of antibiotic compounds in both plant and animal systems. Indeed, antibiotic compounds have been a cornerstone of clinical medicine since the second half of the 20th century. However, onset of antibiotic resistance in bacteria is an increasing crisis as both the range of microbial antibiotic resistance in clinical settings expands, and the pipeline for development of new antibiotics contracts. This problem is compounded by the global genomic scope of the antibiotic resistome, such that antibiotic resistance spans a continuum from genes in pathogens found in the clinic to those of benign environmental microbes.

Multidrug-resistant (MDR) infections caused by antibiotic-resistant bacteria are threatening our ability to treat common infections, causing an estimated 20 billion dollars in direct healthcare costs. The gradual increase in resistance rates of several important pathogens, including methicillin-resistant Staphylococcus aureus, vancomycin-resistant Enterococcus, multidrug-resistant Pseudomonas aeruginosa, imipenem-resistant Acinetobacter baumannii, and third-generation cephalosporin-resistant Escherichia coli and Klebsiella pneumonia, poses a serious threat to public health. Extended-spectrum β lactamase producing pathogens and MRSA are endemic in many hospitals worldwide.

One proposed solution is the utilization of engineered RNA-based molecules. For example, the use of asRNA as highly specific antibacterial drugs has been broadly explored in recent decades. Antisense RNA (asRNA) technology employs production of an RNA molecule which is complementary and hybridizes to a targeted mRNA. As a result of the hybridization of the asRNA to the targeted mRNA, the mRNA is unable to serve as template for protein translation, therefore asRNA-mRNA interaction leads to elimination or reduction of levels of the mRNA encoded protein in the bacteria. In addition, the targeted mRNA may be hydrolyzed by RNases, resulting in post-transcriptional gene silencing. One of the greater obstacles for practical application of asRNA as antibacterial treatments, however, has been the mode of production and delivery of asRNA to infection sites. The challenge has been how to continuously produce and deliver sufficient quantities of asRNA over a long period of time to silence the targeted essential gene in the pathogen at very low or no cost.

As such, there exists a need for a novel solution to the aforementioned technical and practical problems. Indeed, the foregoing problems regarding the control of bacterial pathogens may represent a long-felt need for an effective—and economical—solution to the same. While implementing elements may have been available, actual attempts to meet this need may have been lacking to some degree. This may have been due to a failure of those having ordinary skill in the art to fully appreciate or understand the nature of the problems and challenges involved. As a result of this lack of understanding, attempts to meet these long-felt needs may have failed to effectively solve one or more of the problems or challenges identified herein. These attempts may even have led away from the technical directions taken by the present inventive technology and may even result in the achievements of the present inventive technology being considered, to some degree an unexpected result of the approach taken by some in the field. As will be discussed in more detail below, the current inventive technology overcomes the limitations of traditional bacterial pathogen control systems.

### BRIEF SUMMARY OF THE INVENTION

The present invention relates to the utilization of genetically modified donor bacteria that may be configured to produce certain asRNA polynucleotides that may target specific bacterial genes and/or their products (RNA, proteins) in plant and/or animal systems. These asRNA polynucleotides may inhibit or reduce the expression of certain genes and/or cause the impairment or degradation of gene products in a disease-causing agent. The invention may comprise novel techniques, systems, and methods for controlling pathogenic bacteria, viruses, fungi and/or protozoa in eukaryotic hosts.

One aim of the current inventive technology may include novel systems, methods and compositions for the transbiotic regulation of bacterial gene expression in a recipient pathogenic bacterium by asRNA. One embodiment of the invention may include the effective expression of high levels of asRNA in a donor bacterium species harbored in the host. In certain embodiment, this donor bacterium may be an enteric, and endophytic, and/or a symbiotic bacterium species genetically engineered to express one or more heterologous asRNA polynucleotides.

Another aim of the current invention may include the production of heterologous asRNA in a donor bacterium that may further be delivered to an acceptor bacterium, more specifically a pathogenic bacterium. These heterologous asRNA polynucleotides may target specific genes and their RNA and/or protein products that may be unique and/or restricted to a target bacterial pathogen. Such heterologous asRNA polynucleotides can be fully complementary, or contain mismatches in relation to their targets; both aspects can induce degradation of their targets or impair their translation, making them unavailable for accomplishing their function.

Yet another aim of the current invention may include the suppression of targeted gene expression in the recipient bacteria, resulting in the suppression of bacterial populations and/or pathogenic activity of the bacteria in a host eukaryotic organism.

Another aim of the present invention may include the generation of one or more plasmids and/or bacterial artificial chromosomes (BACs) that may encode one or more heterologous asRNA polynucleotides. An additional aim may include integration of specific genetic elements encoding one or more asRNA into the genome of a pathogen. An additional aim of the invention may be to produce genetic constructs that may produce non-coding RNA molecules, such as the aforementioned heterologous asRNA polynucleotides, by a constitutive, inducible, heterologous, or homologous gene promoter/terminator pair in the donor bacterium strain. Yet another aim of the present invention may include the co-expression of certain proteins or other factors that may protect the non-coding RNA molecule from degradation.

An additional aim of the present invention may include the development of genetically modified auxotrophic bacterial strains that may produce heterologous asRNA polynucleotides that may further be more efficiently delivered to a target pathogen via nanotubes.

Another aim of the present invention may include novel biocontrol strategies for various organisms, including additional animal and plant species. Another aim of the present invention may include, in a preferred embodiment, novel biocontrol strategies for aquaculture populations. In this embodiment, the inventive technology includes various cross-kingdom mechanisms for the knockdown of essential pathogen genes in aquatic animals grown in aquaculture systems. This may be accomplished through the introduction of engineered microorganisms into aquaculture animal populations that express specific heterologous asRNA polynucleotides that may downregulate and/or suppress selected pathogen essential genes.

Another aim of the invention may be the generation of genetically modified symbiotic and/or probiotic bacterial strain that may express one or more heterologous asRNA polynucleotides. In a certain embodiments, shrimp probiotic bacteria, may be genetically modified to express one or more inhibitory RNA molecules directed to essential pathogen genes, preferably in Vibrio sp.

Another aim of the invention present inventive technology may include systems and methods for introducing heterologous asRNA polynucleotides into a target host through infection by genetically engineered donor microorganisms. In one embodiment, the invention may provide for genetically engineered microorganisms that may express one or more heterologous asRNA polynucleotides within a target organism and may be directed to downregulate expression of essential genes in a disease-causing pathogen. Such target organisms may include aquatic animals, aquatic animals in aquaculture systems as well as other vertebrate and invertebrate animals generally.

Additional aims of the inventive technology will become apparent from the figures and descriptions contained herein below.

### BRIEF DESCRIPTION OF THE FIGURES

The accompanying figures, which are incorporated into and form a part of the specification, illustrate one or more embodiments of the present invention and, together with the description, serve to explain the principles of the invention. The drawings are only for the purpose of illustrating one or more preferred embodiments of the invention and are not to be construed as limiting the invention. In the figures:

FIG. 1—AsRNA inhibits GFP fluorescence. A) Reduction of fluorescence level in E. coli HT-115-pGFP strain co-cultivated with E. coli HT-27 strain expressing asRNA-GFP compare to co-cultivation with bacteria expressing unspecific asRNA-COP1 (ns); B) Reduction in fluorescence level in Ag1-pAD-43-25 strain co-cultivated with E. coli HT-27 strain expressing either asRNA-GFP (as-gfp1), compare to co-cultivation with unspecific asRNA-COP (ns).

FIG. 2—Potential effects of asRNA blocking expression of the dam gene. Decrease in methylation of origin/DnaA promoter region leads to disruption of the DNA replication regulation loop and inhibition of Vibrio cell division. Repression of Dam expression inhibits biofilm formation by unknown mechanisms that may include transcriptional inhibition of specific gene promoters resulting in a slowdown of cell growth.

FIG. 3—Effect of Vibrio co-growth with asRNA-Dam expressing AG1 on Vibrio fitness and biofilm formation. AsRNA donor (Enterobacter Ag1) and acceptor (Vibrio Rif^{R}) bacteria were co-inoculated and grown together at various conditions. Cell numbers of rifamycin resistant bacteria (Vibrio) were determined by plating its serial dilutions. A) Co-growth with Ag1-asRNADam leads to 3 fold decrease in Vibrio cell count in liquid cultures (N=24); B) Co-growth with Ag1-asRNA-Dam leads to 1.5 folds decrease in Vibrio cell count on the agar surface (N=8); C) Biofilms formed after 24 h growth in microtiter plate were stained by crystal violet and scored. Co-growth with Ag1-asRNA-Dam leads to 2 fold decrease in biofilm formation.

FIG. 4—Effect of co-growth Ag1-asRNA-Dam on DNA methylation of Vibrio chromosomal DNA. A) Adenines of Vibrio DNA are heavily methylated; B) Co-growth with asRNA-Dam expressing Ag1 decreases 6^{m}A content of Vibrio DNA. In control experiments Vibrio were co-grown with Ag1 expressing non-specific RNA, e.g. asRNA-GFP; C) Results of analysis of changes in Vibrio DNA methylation in response to co-growth with Ag1-asRNA-Dam. Co-growth with Ag1-asRNA-Dam leads to 30% decrease in 6^{m}A content in Vibrio DNA (N=16).

FIG. 5—Co-growth of Vibrio with Ag1-asDam results in decrease of 6^{m}A DNA methylation levels of Vibrio chromosome I replication origin (oriC). A) Schematic drawing of dam (DpnI/MboI) methylation sites in the vicinity of oriC/dnaA promoter. Mapped below the oriC diagram are PCR fragments used in qPCR analysis. Oligonucleotides used in the analysis are presented in Table 4; B) Decrease of 6^{m}A DNA methylation status of Vibrio oriC in the presence of as Dam leads to higher cleavage by MboI restriction enzyme—cuts unmethylated DNA only—and consequently lower amplicon accumulation compared to control (N=8); C) In contrast, oriC DNA is more sensitive to digestion by DpnI—that cut 6^{m}A methylated DNA only—in control samples as a result of higher DNA methylation levels compared to Vibrio oriC in the presence of Ag1-asDam (N=8).

FIG. 6—qRT-PCR analysis of expression of dam and dnaA genes. A) Schematic drawing of Vibrio dam and dnaA gene with mapped PCR products sites; B) Specificity of the oligonucleotides used in the assay to Vibrio DNA; C) Co-growth of Vibrio with Ag1-asDam leads to significant decrease in dam and dnaA mRNAs (N=6).

FIG. 7—qRT-PCR analysis of dam expression in Vibrio living in the intestines of C. elegans fed Ag1-asRNA-Dam or Ag1-asRNA-GFP shows reduction of dam RNA levels as a result of post-transcriptional regulation by asRNA-Dam RNA.

FIG. 8—AsRNA expressing cassette and plasmid. A) asRNA-expressing plasmid map; B) asRNA-expressing cassette design; C) asRNA-Dam antisense RNA—hairpin asRNA structure with asRNA-Dam in the loop. Folding was performed by Mfold application at Mfold web server.

FIG. 9—asRNA-Dam alignment to dam mRNA.

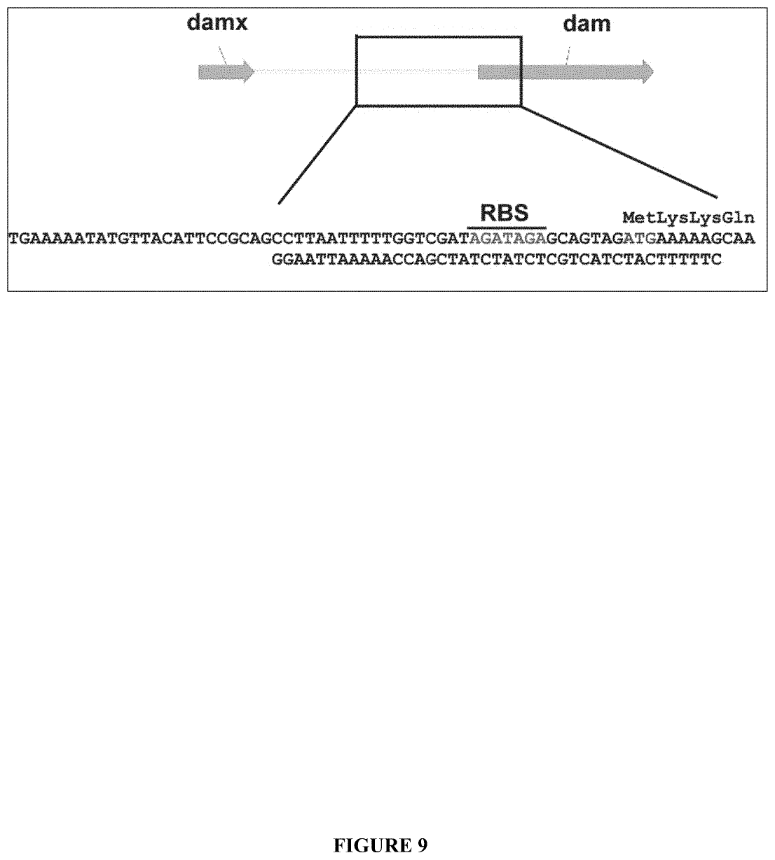

FIG. 10—Diagram depicting mode of action of a generalized asRNA expressed in an engineered bacteria.

FIG. 11—Diagram of generalized quorum sensing pathway in Vibrio harveyi.

FIG. 12—Diagram of exemplary CRISPR/Cas9 system.

### MODE(S) FOR CARRYING OUT THE INVENTION(S)

The present invention includes a variety of aspects, which may be combined in different ways to generally describe the novel systems, methods and compositions related to transbiotic regulation of bacterial gene expression in a recipient pathogenic bacterium by asRNA expressed and delivered by a donor bacterium. The following descriptions are provided to list elements and describe some of the embodiments of the present invention. These elements are listed with initial embodiments, however it should be understood that they may be combined in any manner and in any number to create additional embodiments. The variously described examples and preferred embodiments should not be construed to limit the present invention to only the explicitly described systems, techniques, and applications. Further, this description should be understood to support and encompass descriptions and claims of all the various embodiments, systems, techniques, methods, devices, and applications with any number of the disclosed elements, with each element alone, and also with any and all various permutations and combinations of all elements in this or any subsequent application.

The inventive technology may comprise systems and methods to control the virulence of specific bacterial or other pathogens by selective inactivation of pathogenic, essential or other target genes. This targeted gene inactivation may be accomplished by the expression and delivery of heterologous asRNA molecules from a donor bacterium to a target host pathogen. In one preferred embodiment, one or more donor bacterial species or strains may be genetically engineered to express heterologous asRNA molecules that may act to regulate and/or inhibit gene expression in target disease-causing agents.

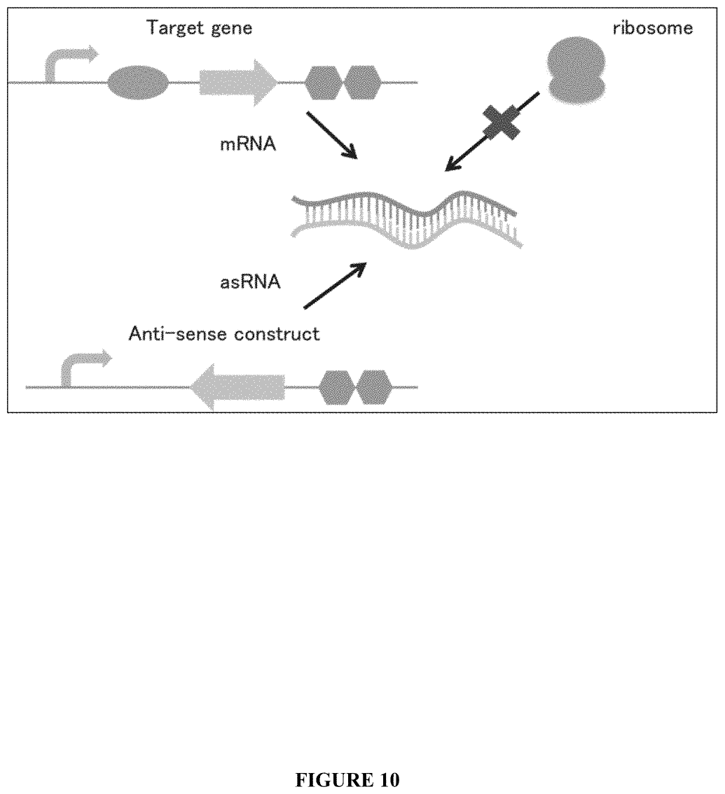

As generally shown in FIG. 10, asRNA may include a non-coding single-stranded RNA molecule that may exhibit a complementary relationship to a specific messenger RNA (mRNA) strand transcribed from a target gene. Additional embodiments may include asRNA having one or more mismatches in relation to their target mRNA. Regardless of the homology between the mRNA and asRNA, in this embodiment, the asRNA may physically pair with, and bond to, the complementary mRNA. This complementary binding may inhibit translation of a complementary mRNA by base pairing the RNA molecules and thereby physically obstructing, or sterically hindering the translation machinery.

It should be noted that when referring to asRNA being complementary, it means that the polynucleotide for use in antisense suppression may correspond to all or part of the complement of the sequence encoding the target polypeptide, all or part of the complement of the 5′ and/or 3′ untranslated region of the target polypeptide transcript, or all or part of the complement of both the coding sequence and the untranslated regions of a transcript encoding the target polypeptide. A complementary nucleic acid molecule is that which is complementary to an mRNA transcript of all or part of a target nucleic acid molecule. In addition, the antisense polynucleotide may be fully complementary (i.e., 100% identical to the complement of the target sequence) or partially complementary (i.e., less than 100% identical to the complement of the target sequence) to the target sequence.

Antisense suppression may be used to inhibit the expression of multiple proteins in the same cell. Furthermore, portions of the antisense nucleotides may be used to disrupt the expression of the target nucleic acid molecule. Generally, antisense sequences of at least 10 nucleotides, 20 nucleotides, 50 nucleotides, 100 nucleotides, 200 nucleotides, 300, 500, 550, 500, 550, or greater, and any amount in-between, may be used. The sequence may be complementary to any sequence of the messenger RNA, that is, it may be proximal to the 5′-terminus or capping site, downstream from the capping site, between the capping site and the initiation codon and may cover all or only a portion of the non-coding region, may bridge the non-coding and coding region, be complementary to all or part of the coding region, complementary to the 3′-terminus of the coding region, or complementary to the 3′-untranslated region of the mRNA.

The antisense sequence may be complementary to a unique sequence or a repeated sequence, so as to enhance the probability of binding. Thus, the antisense sequence may be involved with the binding of a unique sequence, a single unit of a repetitive sequence or of a plurality of units of a repetitive sequence. Methods of preparing antisense nucleic acid molecules are generally known in the art.

As such, in certain embodiments, the present invention may include systems, methods and compositions to inhibit the expression of a nucleic acid molecule of the disease-causing agent, or in some embodiments, the nucleic acid molecule of the disease-causing agent. When referring to inhibiting expression of a target gene, it is meant that expression of the nucleic acid molecule is inhibited, disrupted, or otherwise interfered with such that the eukaryotic recipient, or target host is protected from the disease. Inhibiting expression of a target gene may also generally refer to translation of the nucleic acid molecule being inhibited, disrupted, or otherwise interfered with such that the eukaryotic recipient, or target host is protected from the disease. Inhibiting expression of a target gene, may also mean that expression of the nucleic acid molecule, such as a asRNA polynucleotide, inhibits, disrupts, or otherwise interferes with the expression or translation of an essential gene in a pathogen that the eukaryotic recipient, or target host exhibits lower infection rates, transmission rates, pathogen loads, or disease symptoms that WT hosts.

As noted above, in one embodiment, the invention may include the use of asRNA that is complimentary to a nucleic acid molecule of a target gene in a disease-causing agent. Antisense RNA is RNA that is complementary to a target, usually a messenger RNA (mRNA) of a target nucleic acid molecule. By antisense is intended to mean a sequence that is in inverse orientation to the 5′-to-3′ normal orientation of the target nucleic acid molecule. In a preferred embodiment, a donor bacterium may be genetically modified to express a heterologous asRNA. This expression may be part of an expression vector, and may be part of an expression cassette and may further be operably linked to an expression control sequence(s). This genetically modified donor bacterium may be introduced to a target host and express the targeted heterologous asRNA which may be exported from the donor bacterium and be taken-up into the target disease-causing agent, which in this embodiment may be a pathogenic bacteria. The heterologous asRNA, being delivered to the recipient pathogenic bacteria, may prevent normal expression of the protein encoded by the targeted nucleic acid molecule. This may result in the interference with the disease-causing agent's lifecycle, ability to replicate and/or pathogenicity, thus providing an effective antibacterial delivery system. In this embodiment, the donor bacterium may be a symbiotic bacterium strain that may persist in the target host and provide continuing expression of heterologous asRNA, thus providing on-going production in the host target to counter the disease-causing agent. In additional embodiment, the donor bacterium may be a probiotic, or probiotic-like bacteria that may persist in the target host and express and deliver heterologous asRNA to a recipient bacterial pathogen for a period of time. In this manner, multiple and sequential exposures of the target host to a probiotic, or probiotic-like bacteria may effectively deliver heterologous asRNA, but not persist permanently within the target host.

In another preferred embodiment, a genetically modified donor bacterium may be introduced to a target host that has not been exposed to a target disease-causing agent and may express the targeted heterologous asRNA which may be exported from the donor bacterium into the target host's cellular and/or intracellular environment. The heterologous asRNA, being delivered to the recipient host may act as a prophylactic vaccine such that when the target disease-causing agent, such as a pathogenic bacteria, is introduced to the target host, heterologous asRNA prevents normal expression of the protein encoded by the targeted nucleic acid molecule and may prevent the ability of the disease-causing agent to colonize or affect the target host. In this embodiment, the donor bacterium may be a symbiotic bacterium strain that may persist in the target host and provide continuing expression of heterologous asRNA, thus providing on-going prophylactic vaccine production in the host imparting a level of immunity to the target disease-causing agent.

Additional embodiments may include asRNA-induced gene inactivation of one, or a plurality, of target genes. For example, in one preferred embodiment, gene inactivation may be directed to one or more pathogen genes that are essential to virulence, coat proteins, metabolic activity, infection pathways and/or energy-production and the like. While provided in an exemplary model, a target gene may include one or more genes that are responsible for a bacteria's pathogenicity, or the capacity to cause a disease condition in the host. Examples of such bacterial target genes may also include one or more virulence factors. Virulence factors may help bacteria to: 1) invade the host, 2) cause disease, and/or (3) evade host defenses. In one preferred embodiment, a bacteria strain or species may be modified to express asRNA that may exhibit a complementary relationship to a specific messenger RNA (mRNA) strand transcribed from a target virulence factor gene. Examples of such virulence factors may include, but not be limited to:
Adherence Factors: This group may include genes that help a bacterial pathogens adhere to certain cells;
Invasion Factors: This group may include genes for surface components that allow the bacterium to invade host cells;
Capsules: This group may include genes for structural capsules that may protect bacteria from opsonization and phagocytosis;
Endotoxins: This group may include genes for several types of toxic lipopolysaccharides that may elicit an immune response;
Exotoxins: This group may include genes for several types of protein toxins and enzymes produced and/or secreted from pathogenic bacteria. Major categories include cytotoxins, neurotoxins, and enterotoxins; and
Siderophores: This group may include genes for several types of iron-binding factors that allow some bacteria to compete with the host for iron, which is bound to hemoglobin, transferrin, and lactoferrin.

In one embodiment, the invention may include identification of a target gene in a disease-causing agent. In this preferred embodiment, the target gene may include an essential gene of a disease-causing agent, meaning that the inhibition, disruption, or interference with in the expression and/or translation of one or more essential genes results in the reduction in the number of disease-causing agents, amelioration of pathogenicity of the disease-causing agent, interruption in the disease-causing agent's life-cycle, ability to colonize the eukaryotic host, evade a specific or general immune response in the host, or cause a disease state.

In one embodiment, the heterologous asRNA, directed to a nucleic acid sequence in the disease-causing agent which is to be expressed or inhibited (target nucleic acid molecule or target gene), may either express, inhibit, or compete for binding sites with any such target nucleic acid molecule which, when administered, results in protection to the eukaryotic host from the disease causing agent.

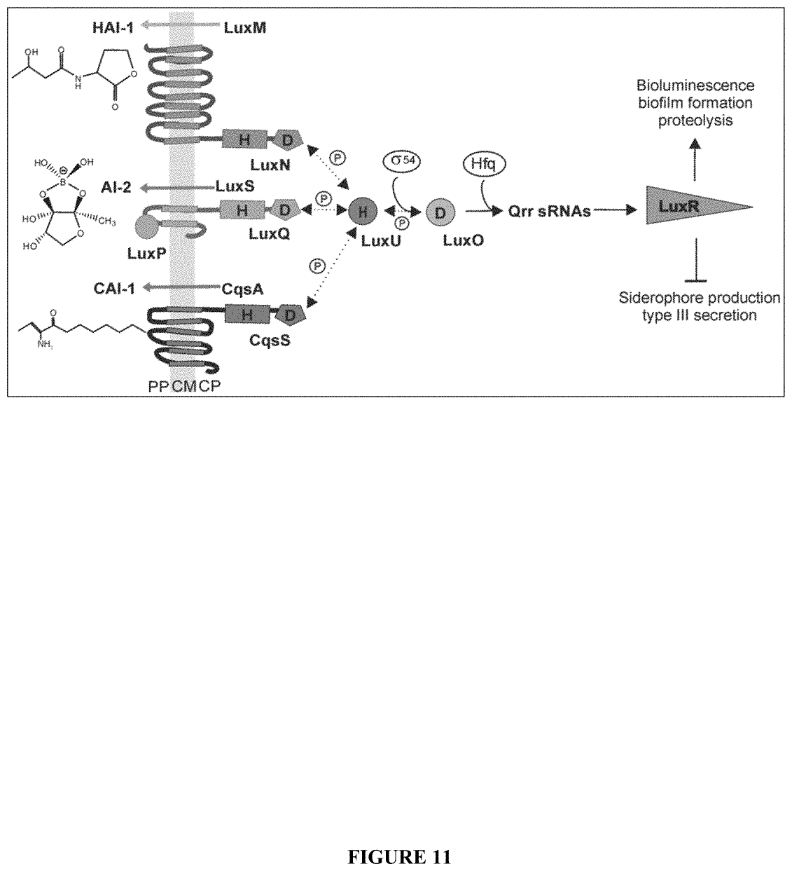

In one embodiment, the invention may include the generation and delivery of a heterologous asRNA directed to one or more target genes in Vibrio harveyi by a donor symbiotic bacterium. In a preferred embodiment, one or more target genes may be involved in mechanisms of quorum-sensing and the formation of biofilms. (See FIG. 11). Generally, quorum-sensing describes a system of stimuli and response correlated to bacterial population density. Quorum sensing may allow bacteria to constantly produce and excrete low-molecular-weight signaling molecules, generally referred to as autoinducers (AIs), into the surrounding environment. As the number of bacteria increase, so does the concentration of AIs. At a defined threshold of AI concentration, the bacterial population may express a synchronized, AI-specific response—usually a phenotype, such as virulence, light production or biofilm formation, which is more effective when deployed by a group of cells rather than a single bacterium. Such quorum sensing responses can greatly enhance bacterial pathogens virulence, as well as make it more difficult to arrest microbial growth through antibiotics or other chemical means, as is the case with bacterial biofilms.

In one specific embodiment, an asRNA may be generated that may be fully or partially complementary to the mRNA from one or more genes that provide for the quorum sensing mechanism in a target bacterial pathogen. In one preferred embodiment, a species or strain of bacteria may be modified to produce an asRNA that may be complementary to the mRNA of one or more AI genes in Vibrio, some species of which are known pathogens of shrimp and other animal hosts. These target AI genes may include HAI-1, AI-1 and/or CAI-1. As can also be seen below, additional gene targets involved in Vibrio harveyi's quorum-sending pathway may also be targets with complementary asRNA, such as CqsA, LuxM, LuxS, and LuxP.

According to one aspect of the present invention there is provided a method of controlling a pathogenically infected organism, the method comprising administering to a target host organism, which in a preferred embodiment may include aquatic organisms, a nucleic acid agent comprising a nucleic acid sequence which specifically downregulates an expression of at least one essential target pathogen gene product, wherein downregulation of the expression of the at least one essential target pathogen gene product in the target host rendering the target host protected from the pathogen-caused disease state. In one preferred embodiment, such a nucleic acid agent may include an asRNA polynucleotide identified as SEQ ID NO 1, or a homolog and/or ortholog thereof. Additional embodiments may include any nucleic acid that spans a region of greater than average homology between an essential target genes of various strains of a disease-causing pathogen. One preferred embodiment may include any nucleic acid that spans a region of greater than average homology between the essential target genes, of various strains of a Vibrio. In the example of a Vibrio harveyi disease causing agent, this may include, as shown generally below in the region encoding the dam gene identified as SEQ ID NO. 3, among others.

In one embodiment, the present invention includes the generation of a novel system for the control of disease-causing pathogens. The invention may specifically include a system configured to deliver to a pathogen-infected, or a pathogen susceptible host, one or more heterologous RNA polynucleotides configured to inhibit expression of one or more essential genes in said pathogen. In one embodiment, the invention may include one or more genetically engineered microorganisms, that may preferably be symbiotic and/or endosymbiotic with a host, and further configured to deliver one or more heterologous RNA molecules, such as asRNA polynucleotides, to pathogen/disease-causing agents. In a preferred embodiment, the invention may include one or more genetically engineered symbiotic bacteria configured to deliver one or more asRNA molecules to pathogenic bacteria in a host organism. In a preferred embodiment, the invention may include one or more genetically engineered symbiotic bacteria configured to deliver one or more asRNA molecules to pathogenic bacteria in an aquatic host organism, such as shrimp or other organisms commonly raised through aquaculture.

In another preferred embodiment, the current inventive technology may extend this technology to symbiotic microorganisms that persist in the tissues, offspring and/or eggs of a host throughout their development and into the adult stage. In this manner, genetically modified symbiotic microorganisms may produce and deliver asRNA molecules continuously to target pathogens such as Vibrio. This may be used to treat a disease-condition in an already infected host, and/or immunize a susceptible host population.

The present invention may further include one or more vectors for inhibiting the expression of multiple pathogen genes, wherein the vector comprising one, or a plurality of heterologous asRNA polynucleotides that may correspond to one or more select pathogen genes. This embodiment may include the use of a plasmid expression system. In some embodiments, this plasmid may have one or more expression cassettes, including: at least one gene suppressing cassette containing a polynucleotide operably linked to an expression control sequence(s), wherein the polynucleotide encodes a heterologous asRNA molecule configured to reduce expression of a target pathogen gene as generally described herein.

A preferred embodiment of the present invention includes a vector for modulating multiple pathogen genes, wherein the vector comprising one or a plurality of asRNAs may correspond to one or more select host genes. This embodiment may include the use of a plasmid expression system. In some embodiments, this plasmid may have one or more expression cassettes, including: at least one gene suppressing cassette containing a polynucleotide operably linked to an expression control sequence(s), wherein the polynucleotide encodes a heterologous asRNA molecule configured to reduce expression of a target pathogen gene as generally described herein.

The present invention also includes a vector for inhibiting the expression of disease-causing agent gene in a host, wherein the vector comprises at least one gene suppressing cassette containing a polynucleotide operably linked to an expression control sequence(s), wherein the polynucleotide encodes an asRNA molecule that reduces expression of a target pathogen gene within the host by RNA interference. In one embodiment, the polynucleotide encoding the asRNA comprises the nucleotide sequence of SEQ ID NO. 1. Examples of suitable promoters for gene suppressing cassettes include, but are not limited to, T7 promoter, b1a promoter, U6 promoter, pol II promoter, E11 promoter, and CMV promoter and the like. Optionally, each of the promoter sequences of the gene promoting cassettes and the gene suppressing cassettes can be inducible and/or tissue-specific.

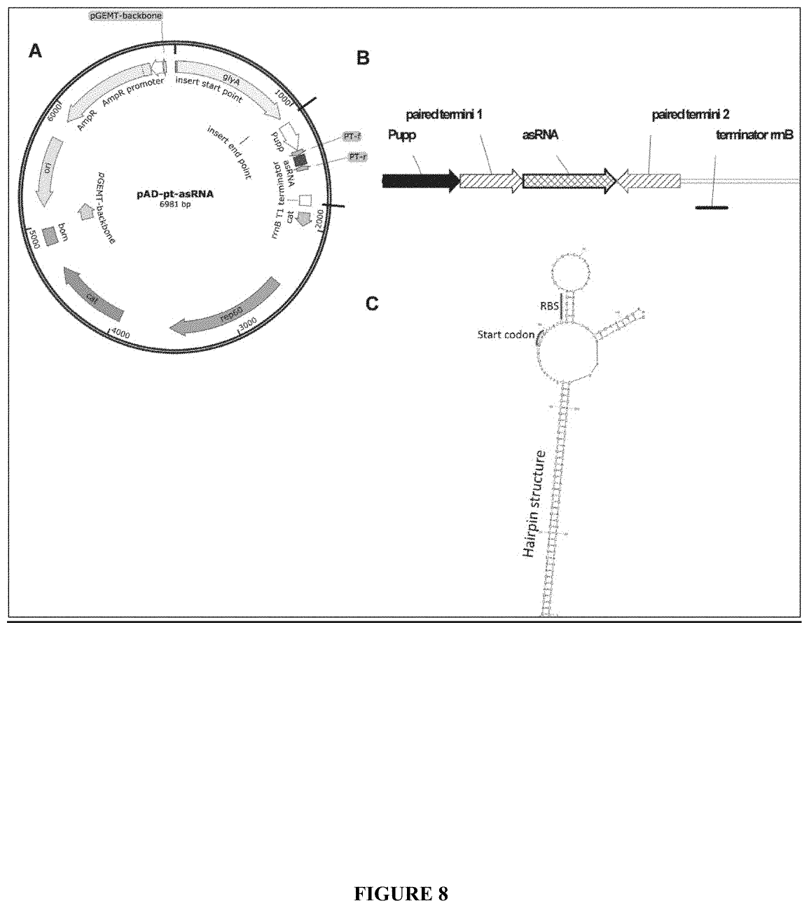

The asRNA molecule, in this embodiment identified as SEQ ID NO. 1, may be partially self-complementary and, therefore, form a stem and loop structure. (See FIG. 8C) The sense region and antisense region of the RNA duplex contain one or more mismatches, such that a bulge or secondary structure (such as a hairpin structure) may be formed. The RNA duplex contains within the range of about 4 to about 23 nucleotide base pair mismatches. More preferably, the RNA duplex contains within the range of about 7 to about 9 nucleotide base pair mismatches. In yet another embodiment, asRNA loop contains within the range of about 50-200 bp, or 59 long bp and ˜90 bp long loops.

In further aspects, the present invention includes methods of administering a therapeutically effective amount of one or more genetically modified donor bacteria expressing a heterologous asRNA polynucleotide. In one embodiment, this therapeutically effective amount may be the amount of bacteria, or the amount of heterologous asRNA polynucleotide expressed by a donor genetically modified bacteria that may be transported out of the donor and taken-up by a target pathogen to ameliorate, reduce or eliminate a disease condition.

In another embodiment, this therapeutically effective amount may be the amount of genetically modified bacteria, or the amount of heterologous asRNA polynucleotide expressed by a donor genetically modified bacteria that may be transported out of the donor such that the host has increased resistance to infection by a later introduced pathogen.

In another embodiment, this therapeutically effective amount may be the amount of genetically modified donor bacteria that can colonize, or become endemic within a population of target hosts through vertical and/or horizontal transfer.

In one embodiment, the present invention may include methods of administering a therapeutically effective amount of a genetically modified bacterium, configured to express heterologous asRNA polynucleotide, may target an essential target gene in Vibrio and may be identified as SEQ ID NO. 1.

In one embodiment, the present invention may include methods for treating and/or preventing the formation of bacterial biofilms. In this embodiment, the method may include the step of administering a therapeutically effective amount of a genetically modified bacteria, configured to express heterologous asRNA polynucleotide, may target an essential target gene in Vibrio that is involved in biofilm production and may be identified as SEQ ID NO. 1.

In one embodiment, the present invention may include methods of administering a therapeutically effective amount of a genetically modified bacteria, configured to express heterologous asRNA polynucleotide, may target an essential target gene in Vibrio that is involved in DNA methylation and may be identified as SEQ ID NO. 1.

Alternative embodiments of the present invention may include a novel in vitro and/or in vivo method to select symbiotic bacteria that may be utilized in an effective system of pathogen gene suppression. In particular, another aim of the present invention may include a novel in vitro and/or in vivo method to select symbiotic host bacteria that may be utilized in an effective system of pathogen gene suppression. These symbiotic host bacteria may be non-pathogenic in humans, and further have culturability, transformability, plasmid mobilization, and be able to able to secrete target nucleic acids, such as asRNA and the like, endemic or able to become endemic in host populations, dispersible, for example through aerosolization, able to survive in the environment and be eaten or taken up by hosts at all stages of life preferably.

In another aspect, the present invention includes methods for producing the vectors of the present invention. In yet another aspect, the present invention includes methods for producing the transformed or genetically modified microorganisms of the present invention, for example through transformation with a recombinant plasmid.

Another embodiment of the present invention may include a cell, such as a genetically modified microorganism, configured to express a heterologous nucleic acid agent, such as a asRNA, or the nucleic acid construct, such as a plasmid, of some embodiments of the invention. In one preferred embodiment, the present invention may include a genetically modified bacteria, configured to express a heterologous asRNA polynucleotide. In a further preferred embodiment, heterologous asRNA polynucleotide may target an essential target gene in Vibrio and may be identified as SEQ ID NO. 1.

Another embodiment of the present invention may include a cell comprising the isolated nucleic acid agent, such as a asRNA, or the nucleic acid construct, such as a plasmid, of some embodiments of the invention wherein the cell is selected from the group consisting of a bacterial cell, an algae cell, a symbiotic bacteria, and a cell of a water surface microorganism. According to an aspect of some embodiments of the present invention, there is provided an ingestible compound comprising the cell of some embodiments of the invention.

In another preferred embodiment, a species or strain of bacteria may be modified to produce an asRNA that may be complementary to the mRNA encoding DNA adenine methylase (Dam) in Vibrio harveyi. These modified bacteria may include strains or species that are part of the normal flora of shrimp, and or symbiotic and/or endosymbiotic with a target host, such as shrimp or other aquatic organisms. Upon introduction, these genetically engineered bacteria maybe taken up by the shrimp and become part of the normal flora.

In this embodiment, asRNA expressed in a donor bacterium, such as E. coli or Enterobacter, may suppress the expression of the dam, or other essential gene in Vibrio in a target host. In another embodiment, asRNA-Dam expressed in a donor bacterium, identified as SEQ ID NO. 1, may decrease Vibrio fitness and also generate a pronounced decline in biofilm formation or pathogenesis. As detailed below, the decrease in Vibrio fitness is directly related to a reduction of Dam expression in the recipient Vibrio cells as indicated by the observation that: 1) Vibrio DNA is 30% less methylated when co-cultivated with bacteria expressing asRNA-Dam; 2) The Vibrio replication origin oriC and promoter of dnaA, critical elements in the initiation of DNA replication, were 2-folds less methylated than in controls not exposed to bacteria expressing asRNA-Dam; 3) Expression of Vibrio dam gene was also decreased 2-fold relative to controls; 4) Expression of the Vibrio dnaA gene was decreased 3-fold relative to controls; 5) Expression of Vibrio dam gene was decreased 6-fold when exposed to Enterobacter Ag1 expressing asRNA-Dam in the model animal organism. Such results demonstrate the ability of the current invention to control disease and biofilm generation by targeted production and delivery of asRNA from a donor to a recipient bacterium in a host organism.

As noted above, the delivery of heterologous asRNA, may be accomplished through the introduction of genetically modified host-specific donor microorganisms, such as enteric, endophytic, symbiotic or endosymbiotic bacteria. Such genetically modified host-specific microorganisms may include: 1) microorganisms that are part of the target pathogen's normal internal or external bacterial microbiome; 2) microorganisms that have been modified to be capable of colonizing a target animal, plant, tissue, cell or host environment; 3) microorganisms that that are utilized as a food or energy source by the target host; or 4) microorganisms that have been modified to colonize, or transiently persist in the target host as in the case of a probiotic or probiotic-like microorganism, a specific animal, plant, tissue, cell or host environment. As noted above, in one preferred embodiment, the heterologous asRNA donor bacterium may include E. coli, as well as bacterium from the genus Enterobacter. In another preferred embodiment, heterologous asRNA donor bacterium may include one or more enteric bacteria selected from the group identified below in Table 5 below. Naturally, such examples are non-limiting, as any bacterium that is able, and/or configured to stable colonize or enter a symbiotic relationship with the target host, which may act as a donor bacterium.

In one preferred embodiment, donor bacteria may be transformed with artificially created genetic constructs, such as plasmids that may generate heterologous asRNA polynucleotides. Such plasmids may be constructed to be transferrable to other bacteria through conjugation and other means which may allow for widespread distribution of the construct, in some instances. In certain embodiments, asRNA molecules can be encoded on plasmids and/or BACs under the control of a constitutive, inducible, heterologous, or homologous gene promoter/terminator pair in the donor bacteria delivering the heterologous asRNA polynucleotides. In an additional embodiment, genetic constructs for the generation of heterologous asRNA polynucleotides may be integrated into the bacterial genome of the delivery or host bacteria.

In another preferred embodiment, one or more heterologous asRNA polynucleotides may be delivered to a target animal host/population through genetically modified donor bacteria that may naturally colonize the host, or be configured to colonize the host. The donor bacteria may then, in one preferred embodiment, disseminate the genetic constructs expressing the heterologous asRNA polynucleotides to naturally occurring host microorganisms and/or pathogenic bacteria in the surrounding environment. In this embodiment, once colonized in the target host, vertical transmission of the modified bacteria may be passed to the host's progeny, thus naturally replicating the pathogenic bacterial resistance to subsequent generations. Additionally, the modified bacteria may also be horizontally transmitted to the host population at large through the distribution of the modified bacteria into the environment as waste. Such a feature may allow for the one-time, or at least only periodic, administration of the genetically modified bacteria to the host and/or host's environment, generating a significant commercial advantage.

The inventive technology may further comprise methods and techniques to control the levels and timing of the expression of heterologous asRNA polynucleotides in the donor bacteria. In one preferred embodiment, the expression of one or more heterologous asRNA polynucleotides may be under the control of a novel gene switch. This gene switch may be controlled by a switch molecule, which may be a water-soluble and food-grade molecule that can be added to a host organism's environment or a food supply. The presence of this switch molecule may activate, for example heterologous asRNA production. In its absence, asRNA production may not occur, or may only occur at negligible levels.

In certain embodiments, a host-specific or symbiotic donor bacteria may include one or more of the following characteristics: 1) a prevalent bacteria in the target host's microbiome, for example in the gut flora of a target host; 2) culturable outside of the host, for example in a fermenter; 3) no known adverse environmental or health impacts on non-target organisms; 4) capable of being genetically engineered to stably express heterologous asRNA molecules in sufficient quantities to inhibit target gene replication, in at least one, but preferably all, life stages of the host's life cycle; and 5) configured in genetic constructs that may be mobilized into other bacteria within the host.

Additional embodiments of the present invention may include methods and systems to optimize the effectiveness of heterologous asRNA polynucleotides. In one preferred embodiment, asRNA may be co-expressed and/or fused with chaperone proteins to protect the RNA molecules from degradation. Additional preferred embodiments may include the co-expression and/or fusing of secretion tags/moieties that may facilitate secretion and/or uptake of heterologous asRNA polynucleotides, increasing their effectiveness.

Bacterial endoribonucleases, exoribonucleases and RNA degradosomes may degrade non-coding RNA molecules such as asRNA or gRNA. In one embodiment, the inventive technology may include modification of the previously identified delivery bacteria to have decreased expression, or inactivated function or activity of these protein families. This decrease or inactivation in expression and/or activity may inhibit or decrease single-stranded non-coding RNA species degradation. In one preferred embodiment, the previously identified host-specific bacteria may be genetically modified to efficiently express heterologous asRNA polynucleotides in an RNA endoribonuclease, exoribonuclease and/or degradosomes deficient background. In one preferred embodiment, a donor bacterium may lack, or have degraded RNase III function. In this preferred embodiment, these non-coding RNA molecule degradation genes may be knocked out by homologous recombination or other appropriate methods.

Another embodiment of the inventive technology may include systems and methods to facilitate the overexpression of host-specific bacterial genes known to enhance stabilization and/or mobilization of non-coding RNA molecules, such as asRNA and/or gRNA, as well as the mobilization and dissemination of their underlying genetic constructs, such as plasmids. In this preferred embodiment, one or more genes known to stabilize asRNA or mobilize genetic constructs such as plasmids may be overexpressed to enhance their lifetime and facilitate movement within host organism/cell/tissue.

Another preferred embodiment of the present invention may be to provide leaf and root endophytic and ectophytic bacteria that may further be genetically engineered to express non-coding RNA molecules, such as asRNA and gRNA. Non-limiting examples of genetically modified endophytic bacteria may include those in the subphyla: Acidobacteria, Actinobacteria, Alphaproteobacteria, Armatimonadetes, Bacteroides, Betaproteobacteria, Deltaproteobacteria, Firmicutes, Grammaproteobacteria, and TM7. In one preferred embodiment, ectophytic bacteria may be transformed with artificially created genetic constructs, such as plasmids that may generate the heterologous asRNA polynucleotides. Again, such plasmids may be constructed to be transferrable to other bacteria through conjugation which may allow for widespread environmental inoculation in some instances.

In another embodiment, non-coding RNA molecules, such as heterologous asRNA polynucleotides, may be delivered by engineered and/or genetically modified bacteria that induce formation of intracellular connections, especially in non-optimal environmental conditions, or where certain essential nutrients are lacking in the surrounding environment. In this manner, bacteria may form nanotubes to exchange nutrients, genetic material and other chemical signals among connected cells and thus help to distribute metabolic functions within microbial communities. In this embodiment, auxotrophic bacteria may be genetically modified to induce formation of nanotubes which may allow the direct dissemination of asRNA from donor bacteria to target or recipient bacteria. In another embodiment, auxotrophic bacteria may be genetically modified to induce formation of nanotubes which may allow the dissemination of genetic constructs that encode for asRNA to target bacteria which lack the artificial genetic construct. In this configuration, under certain environmental or nutrient-deficient conditions, delivery bacteria may disseminate asRNA and/or the genetic constructs, such as plasmids, that encode an asRNA to other bacteria in the community. This action may help impair the expression of specific target genes among a large population of pathogenic bacteria.

In this embodiment, such genetic constructs may include transcription regulation portions, such as promoters, terminators, co-activators and co-repressors and similar control elements that may be regulated in prokaryotic, as well as eukaryotic systems. Such systems may allow for control of the type, timing and amount of heterologous asRNA polynucleotides, or other non-coding RNA molecules, expressed within the system. Additional embodiments may include genetic constructs that may be induced through outside factors, such as the presence of a specific protein or compound within a cell, such as stress related proteins generated in response to a pathogen, or even the proteins and other precursor compounds generated by pathogens and the like.

In another preferred embodiment, the present inventive technology may include systems and methods whereby genetically transformed leaf and root endophytic and ectophytic bacteria that generate one or more non-coding RNA molecules, such as heterologous asRNA polynucleotides, may be delivered to targeted plant phyla and/or species where the non-coding RNA molecules may be transferred to pathogenic bacteria and inactivate and/or knock-down expression of target pathogenic genes. In certain embodiments, selection of microorganisms, such as bacteria that are known to be specific to one phyla or even a certain species may be utilized. In another embodiment, transcriptional activation and promotion of non-coding RNA molecules may be dependent on the presence of certain factors that may be specific to one phyla, or even a certain plant or herbivore species. For example, in certain embodiment, non-coding RNA, such as asRNA molecules can be encoded on plasmids and/or BACs under the control of a constitutive, inducible, heterologous, or homologous gene promoter/terminator pair in the endophytic or exophytic bacteria delivering the molecules. In additional embodiment, genetic constructs for the generation of asRNA may be integrated into the bacterial genome of the donor, naturally occurring host, and/or target pathogenic bacteria.

In certain embodiments, endophytic or enteric bacteria may be genetically modified to produce non-coding RNA molecules, such as heterologous asRNA polynucleotides, that may target specific genes that confer drug resistance to certain pathogenic bacteria. In the case of a disease condition, a non-coding RNA molecule may interfere with one or more target genes related to bacterial pathogenicity, as well as genes that confer drug-resistance. In this embodiment, treatment of bacterial pathogens in plant and animal systems may be accomplished through the action of the symbiotic bacteria producing heterologous asRNA polynucleotides and/or gRNA that disrupt one or more genes associated with MDR alone, or in conjunction with traditional antibiotics or other pharmacological compounds. Examples of gene targets related to MDR in pathogenic bacteria are provided in Table 6 below. Examples of animal pathogens that may be targeted with the present inventive technology are included in Table 7 below. Such lists are exemplary only, and should not be construed as limiting in any way.

As noted above, in a preferred embodiment, one or more heterologous asRNA polynucleotides may be delivered to a target host/population of shrimp through genetically modified bacteria that may naturally, or be configured to, colonize and/or be symbiotic with the shrimp. In this embodiment, once colonized in the host, vertical transmission of the modified bacteria may be passed to the host's progeny, thus naturally replicating the pathogenic resistance to subsequent generations. In certain embodiments, genetically modified bacteria expressing one or more heterologous asRNA polynucleotides may colonize a shrimp throughout its lifecycle. For example, a genetically modified donor bacteria expressing one or more heterologous asRNA polynucleotides may colonize a shrimp while it is: an egg, a nauplius, a protozoea, a mysis, post-larval stage or an adult. In this embodiment, the colonized bacteria may express heterologous asRNA polynucleotides, that may be directed to be expressed and transported from the donor bacterium and taken up by a recipient pathogen bacteria and inhibit expression or one or more essential genes. Moreover, these colonized bacteria, having permanently and/or temporarily become a part of the host's natural microbiome, may continuously deliver the heterologous asRNA polynucleotides, in one instance via the intestine from the earliest larval stages to the adult stage, providing pathogen-specific mRNA down-regulation of essential pathogen genes throughout the host's lifecycle. In addition, as the donor bacterial vector may be an already naturally occurring part of the host's microbiome, its presence may not pose any risk to the organism, environment or end-consumers.

The inventive technology may include methods and techniques for the generation of host-specific bacteria, and in particular, host-specific enteric or symbiotic bacteria that may act as an appropriate donor vector for heterologous asRNA polynucleotides directed to bacterial pathogens that affect aquatic organisms. As an exemplary model, shrimp may be utilized as a target host. However, as can be appreciated by one of ordinary skill in the art, such methods and techniques may be applied to a variety of different organisms.

The term “aquaculture” as used herein includes the cultivation of aquatic organisms under controlled conditions.

The term “aquatic organism” and/or “aquatic animal” as used herein include organisms grown in water, either fresh or saltwater. Aquatic organisms/animals includes vertebrates, invertebrates, arthropods, fish, mollusks, including, shrimp (e.g., penaeid shrimp, Penaeus esculentu, Penaeus setiferus, Penaeus stylirostris, Penaeus occidentalis, Penaeus japonicus, Penueus vunnamet, Penaeus monodon, Penaeus chinensis, Penaeus aztecus, Penaeus duorarum, Penaeus indicus, and Penaeus merguiensis, Penaeus californiensis, Penaeus semisulcatus, Penaeus monodon, brine shrimp, freshwater shrimp, etc), crabs, oysters, scallop, prawn clams, cartilaginous fish (e.g., sea bream, trout, bass, striped bass, tilapia, catfish, salmonids, carp, catfish, yellowtail, carp zebrafish, red drum, etc), crustaceans, among others. Shrimp include, shrimp raised in aquaculture as well.

The term “probiotic” refers to a microorganism, such as bacteria, that may colonize a host for a sufficient length of time to deliver a therapeutic or effective amount of an heterologous asRNA polynucleotide. A probiotic may include endosymbiotic bacteria, or naturally occurring flora that may permanently to temporarily colonize an animal, such as an aquatic organism. Probiotic organisms may also include algae, and fungi, such as yeast.

Specific examples of bacterial vectors include bacteria (e.g., cocci and rods), filamentous algae and detritus. Specific embodiments of transformable bacterial vectors cells that may be endogenous through all life cycles of the host may include all those listed herein. Additional embodiments may include one or more bacterial strains selected from the examples listed herein. Naturally, such a list is not exclusive, and is merely exemplary of certain preferred embodiments of paratransgenic bacterial strains.

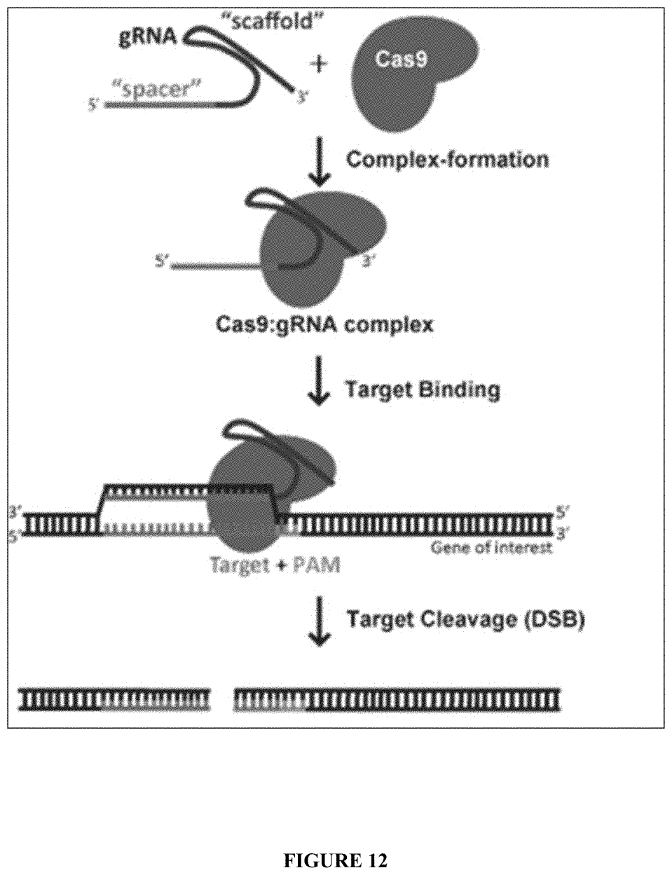

The present invention may include novel systems and methods for the expression of gRNA in a symbiotic donor bacterial species or strain which may be utilized by the CRISPR/Cas9 system to disrupt of target genes in pathogenic bacteria expressing CRISPR/Cas9 genes. Generally, CRISPR/Cas9 may be used to generate a knock-out or disrupt target genes by co-expressing a gRNA specific to the gene to be targeted and the endonuclease Cas9. Generally referring to FIG. 12, CRISPR may consist of two components: gRNA and a non-specific CRISPR-associated endonuclease (Cas9). The gRNA may be a short synthetic RNA composed of a scaffold sequence that may allow for Cas9-binding and a ˜20 nucleotide spacer or targeting sequence which defines the genomic target to be modified. In one preferred embodiment, exemplary bacteria, such as symbiotic, endosymbiotic bacteria may be genetically modified to produce one or more gRNAs that are targeted to the genetic sequence of a pathogenic or other target gene and that can associate with the target bacteria's naturally occurring Cas9 endonuclease. In another preferred embodiment, exemplary bacteria, such as endophytic and/or enteric bacteria may be genetically modified to produce one or more gRNAs that are targeted to the genetic sequence of a pathogenic or other target gene and that can associate with the target bacteria's naturally occurring Cas9 endonuclease.

As used herein, the term “antisense RNA” or “asRNA” refers to an RNAi agent that is a single stranded oligonucleotide. In a typical asRNA, the single strand is complementary to all or a part of the target mRNA. The complementarity of an asRNA may be with any part of the specific gene transcript, i.e., at the 5′ non-coding sequence, 3′ non-translated sequence, introns, or the coding sequence. asRNA may be introduced into a cell to inhibit translation of a complementary mRNA by base pairing to it and physically obstructing the translation machinery. Antisense RNA anneal to a complementary mRNA target sequence, and translation of the mRNA target sequence is disrupted as a result of steric hindrance of either ribosome access or ribosomal read through. The antisense RNA mechanism is different from RNA interference (RNAi), a related process in which double-stranded RNA fragments (dsRNA, also called small interfering RNAs (siRNAs)) trigger catalytically mediated gene silencing, most typically by targeting the RNA-induced silencing complex (RISC) to bind to and degrade the mRNA. Annealing of a strand of the asRNA molecule to mRNA or DNA can result in fast degradation of duplex RNA, hybrid RNA/DNA duplex, or duplex RNA resembling precursor tRNA by ribonucleases in the cell, or by cleavage of the target RNA by the antisense compound itself

As used herein, Vibrio is a genus of Gram-negative, facultative anaerobic bacteria possessing a curved-rod shape, with Vibrio sp. indicating a species within the genus Vibrio. In some embodiments, Vibrio sp. can comprise any one or more of the following Vibrio species, and in all possible combinations: adaptatus, aerogenes, aestivus, aestuarianus, agarivorans, albensis, alfacsensis, alginolyticus, anguillarum, areninigrae, artabrorum, atlanticus, atypicus, azureus, brasiliensis, bubulus, calviensis, campbellii, casei, chagasii, cholera, cincinnatiensis, coralliilyticus, crassostreae, cyclitrophicus, diabolicus, diazotrophicus, ezurae, fischeri, fluvialis, fortis, furnissii, gallicus, gazo genes, gigantis, halioticoli, harveyi, hepatarius, hippocampi, hispanicus, hollisae, ichthyoenteri, indicus, kanaloae, lentus, litoralis, logei, mediterranei, metschnikovii, mimicus, mytili, natriegens, navarrensis, neonates, neptunius, nereis, nigripulchritudo, ordaii, orientalis, pacinii, parahaemolyticus, pectenicida, penaeicida, pomeroyi, ponticus, proteolyticus, rotiferianus, ruber, rumoiensis, salmonicida, scophthalmi, splendidus, superstes, tapetis, tasmaniensis, tubiashii, vulnificus, wodanis, and xuii.

As used herein, the phrase “host” or “target host” refers to a organism or population carrying a disease-causing pathogen, or an organism or population that is susceptible to a disease-causing pathogen. A “host” or “target host” may further include an organism or population capable of carrying a disease-causing pathogen.

As used herein, the terms “controlling” and/or “bio-control” refer to reducing and/or regulating pathogen/disease progression and/or transmission.

As used herein, “vaccine” refers to compositions that result in both active and passive immunizations. Both polynucleotides and their expressed gene products are referred to as vaccines herein. A feed including a treated bacteria configured to express an heterologous RNA polynucleotide may also be a vaccine. Feeding treated feed to an animal may be a vaccination.

As used herein, the phrase “feed” refers to animal consumable material introduced as part of the feeding regimen or applied directly to the water in the case of aquatic animals. A “treated feed” refers to a feed treated with a treated bacteria configured to express an interfering bacteria.

The term “nucleic acid” as used herein, refers to a polymer of ribonucleotides or deoxyribonucleotides. Typically, “nucleic acid or “nucleic acid agent” polymers occur in either single or double-stranded form, but are also known to form structures comprising three or more strands. The term “nucleic acid” includes naturally occurring nucleic acid polymers as well as nucleic acids comprising known nucleotide analogs or modified backbone residues or linkages, which are synthetic, naturally occurring, and non-naturally occurring, which have similar binding properties as the reference nucleic acid, and which are metabolized in a manner similar to the reference nucleotides. Exemplary analogs include, without limitation, phosphorothioates, phosphoramidates, methyl phosphonates, chiral-methyl phosphonates, 2-O-methyl ribonucleotides, and peptide-nucleic acids (PNAs). “DNA”, “RNA”, “polynucleotides”, “polynucleotide sequence”, “oligonucleotide”, “nucleotide”, “nucleic acid”, “nucleic acid molecule”, “nucleic acid sequence”, “nucleic acid fragment”, and “isolated nucleic acid fragment” are used interchangeably herein.

The term “recombinant” when used with reference, e.g., to a cell, or nucleic acid, protein, or vector, indicates that the cell, organism, nucleic acid, protein or vector, has been modified by the introduction of a heterologous nucleic acid or protein or the alteration of a native nucleic acid or protein, or that the cell is derived from a cell so modified. Thus, for example, recombinant cells may express genes that are not found within the native (non-recombinant or wild-type) form of the cell or express native genes that are otherwise abnormally expressed, over-expressed, under expressed or not expressed at all.

The terms “genetically modified,” “bio-transformed,” “transgenic”, “transformed”, “transformation”, and “transfection” are similar in meaning to “recombinant”. “Transformation”, “transgenic”, and “transfection” refer to the transfer of a polynucleotide into the genome of a host organism or into a cell. Such a transfer of polynucleotides can result in genetically stable inheritance of the polynucleotides or in the polynucleotides remaining extra-chromosomally (not integrated into the chromosome of the cell). Genetically stable inheritance may potentially require the transgenic organism or cell to be subjected for a period of time to one or more conditions which require the transcription of some or all of the transferred polynucleotide in order for the transgenic organism or cell to live and/or grow. Polynucleotides that are transformed into a cell but are not integrated into the host's chromosome remain as an expression vector within the cell. One may need to grow the cell under certain growth or environmental conditions in order for the expression vector to remain in the cell or the cell's progeny. Further, for expression to occur, the organism or cell may need to be kept under certain conditions. Host organisms or cells containing the recombinant polynucleotide can be referred to as “transgenic” or “transformed” organisms or cells or simply as “transformants”, as well as recombinant organisms or cells.

The term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature; etc. There are various types of vectors including virus, plasmid, bacteriophages, cosmids, and bacteria.

An “expression vector” is a nucleic acid capable of replicating in a selected host cell or organism. An expression vector can replicate as an autonomous structure, or alternatively can integrate, in whole or in part, into the host cell chromosomes or the nucleic acids of an organelle, or it may be used as a shuttle for delivering foreign DNA to cells, and thus replicate along with the host cell genome. Thus, expression vectors are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette”. In contrast, as described in the examples herein, a “cassette” is a polynucleotide containing a section of an expression vector of this invention. The use of the cassette assists in the assembly of the expression vectors. An expression vector is a replicon, such as plasmid, phage, virus, chimeric virus, or cosmid, and which contains the desired polynucleotide sequence operably linked to the expression control sequence(s).

A polynucleotide sequence is “operably linked to an expression control sequence(s)” (e.g., a promoter and, optionally, an enhancer) when the expression control sequence controls and regulates the transcription and/or translation of that polynucleotide sequence. As used herein, the phrase “gene product” refers to an RNA molecule or a protein. Moreover, the term “gene” may sometime refer to the genetic sequence, the transcribed and possibly modified mRNA of that gene, or the translated protein of that mRNA.

The present teachings contemplate the targeting of homologs and orthologs according to the selected pathogen species, for example species of Vibrio. Homologous sequences include both orthologous and paralogous sequences. The term “paralogous” relates to gene-duplications within the genome of a species leading to paralogous genes. The term “orthologous” relates to homologous genes in different organisms due to ancestral relationship. Thus, orthologs are evolutionary counterparts derived from a single ancestral gene in the last common ancestor of given two species (Koonin E V and Galperin M Y (Sequence-Evolution-Function: Computational Approaches in Comparative Genomics. Boston: Kluwer Academic; 2003. Chapter 2, Evolutionary Concept in Genetics and Genomics. Available from: www.ncbi.nlm.nih.gov/books/NBK20255/) and therefore have great likelihood of having the same function. As such, orthologs usually play a similar role to that in the original species in another species.

Homology (e.g., percent homology, sequence identity+sequence similarity) can be determined using any homology comparison software computing a pairwise sequence alignment. As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 1992, 89(22): 10915-9].

According to a specific embodiment, a homolog sequences are at least 60%, 65%, 70%, 75%, 80%, 85%, 90%, 95% or even identical to the sequences (nucleic acid or amino acid sequences) provided herein. Homolog sequences of any of SEQ ID Nos 1-4 of between 50%-99% may be included in certain embodiments of the present invention.

Downregulating expression of a pathogen gene product can be monitored, for example, by direct detection of gene transcripts (for example, by PCR), by detection of polypeptide(s) encoded by the gene (for example, by Western blot or immunoprecipitation), by detection of biological activity of polypeptides encoded by the gene (for example, catalytic activity, ligand binding, and the like), or by monitoring changes in the host (for example, reduced motility of the host etc.). Additionally, or alternatively downregulating expression of a pathogen gene product may be monitored by measuring pathogen levels (e.g. bacterial levels etc.) in the host as compared to a wild type (i.e. control) host not treated by the agents of the invention.

As used herein, the term “interfering RNA molecules” or “interfering RNA” refers to an RNA polynucleotide which is capable of inhibiting or “silencing” the expression of a target gene in a pathogen. In certain embodiments, an “interfering RNA molecule” or “interfering RNA” may include an asRNA or heterologous asRNA. The inhibitory RNA sequence can be greater than 90% identical or even 100% identical, to the portion of the target gene transcript. Alternatively, the duplex region of the RNA may be defined functionally as a nucleotide sequence that is capable of hybridizing with a portion of the target gene transcript under stringent conditions (e.g., 400 mM NaCl, 40 mM PIPES pH 6.4, 1 mM EDTA, 60 degrees C. hybridization for 12-lb hours; followed by washing). The length of the single-stranded nucleotide sequences complementary to the target gene transcript may be at least about 18, 19, 21, 25, 50, 100, 200, 300, 400, 491, 500, 550, 600, 650, 700, 750, 800, 900, 1000 or more bases. In some embodiments of the invention, the length of the double-stranded nucleotide sequence is approximately from about 18 to about 530, or longer, nucleotides in length.

It will be noted that the asRNA can be defined in terms of the nucleic acid sequence of the DNA encoding the target gene transcript, and it is understood that a asRNA sequence corresponding to the coding sequence of a gene comprises an RNA complement of the gene's coding sequence, or other sequence of the gene which is transcribed into RNA.

For example, in order to silence the expression of an mRNA of interest, synthesis of the asRNA suitable for use with some embodiments of the invention can be selected as follows. First, the mRNA sequence is scanned including the 3′ UTR and the 5′ UTR. Second, the mRNA sequence is compared to an appropriate genomic database using any sequence alignment software, such as the BLAST software available from the NCBI server (wwwdotncbidotnlmdotnihdotgov/BLAST/). Putative regions in the mRNA sequence which exhibit significant homology to other coding sequences are filtered out. Qualifying target sequences are selected as templates for asRNA synthesis. Preferred sequences are those that have little homology to other genes in the genome to reduce an “off-target” effect.

It will be appreciated that the RNA silencing agent of some embodiments of the invention need not be limited to those molecules containing only RNA, but further encompasses chemically-modified nucleotides and non-nucleotides.

According to one embodiment, the asRNA specifically targets a gene selected from the group consisting of SEQ ID NO. 3, or a variant of homolog thereof.

As used herein, the term “heterologous” refers to exogenous, not-naturally occurring within a native cell of the donor, host, pathogen or in a cell in which the asRNA is introduced (such as by position of integration, or being non-naturally found within the cell).

According to a specific embodiment, the vector for the heterologous asRNA polynucleotide, or donor is a bacteria. In other embodiments, the donor is an algae cell. Various algae species can be used in accordance with the teachings of the invention since they are a significant part of the diet for many kinds of hosts that feed opportunistically on microorganisms as well as on small aquatic animals such as rotifers. Examples of algae that can be used in accordance with the present teachings include, but are not limited to, blue-green algae as well as green algae. Specifically, Actinastrum hantzschii, Ankistrodesmus falcatus, Ankistrodesmus spiralis, Aphanochaete elegans, Chlamydomonas sp., Chlorella ellipsoidea, Chlorella pyrenoidosa, Chlorella variegate, Chlorococcum hypnosporum, Chodatella brevispina, Closterium acerosum, Closteriopsis acicularis, Coccochloris peniocystis, Crucigenia lauterbomii, Crucigenia tetrapedia, Coronastrum ellipsoideum, Cosmarium botrytis, Desmidium swartzii, Eudorina elegans, Gloeocystis gigas, Golenkinia minutissima, Gonium multicoccum, Nannochloris oculata, Oocystis marssonii, Oocystis minuta, Oocystis pusilla, Palmella texensis, Pandorina morum, Paulschulzia pseudovolvox, Pediastrum clathratum, Pediastrum duplex, Pediastrum simplex, Planktosphaeria gelatinosa, Polyedriopsis spinulosa, Pseudococcomyxa adhaerans, Ouadrigula closterioides, Radiococcus nimbatus, Scenedesmus basiliensis, Spirogyra platensis, Staurastrum gladiosum, Tetraedron bitridens, Trochiscia hystrix. Anabaena catenula, Anabaena spiroides, Chroococcus turgidus, Cylindrospermum licheniforme, Bucapsis sp. (U. Texas No. 1519), Lyngbya spiralis, Microcystis aeruginosa, Nodularia spumigena, Nostoc linckia, Oscillatoria lutea, Phormidiumfaveolarum, Spinilina platensis.

In a further embodiment, a composition including a genetically modified bacteria configured to express asRNA may be formulated as a water dispersible granule or powder that may further be configured to be dispersed into the environment. In yet a further embodiment, the compositions of the present invention may also comprise a wettable powder, spray, emulsion, colloid, aqueous or organic solution, dust, pellet, or colloidal concentrate. Dry forms of the compositions may be formulated to dissolve immediately upon wetting, or alternatively, dissolve in a controlled-release, sustained-release, or other time-dependent manner. Alternatively or additionally, the composition may comprise an aqueous solution. Such aqueous solutions or suspensions may be provided as a concentrated stock solution which is diluted prior to application, or alternatively, as a diluted solution ready-to-apply. Such compositions may be formulated in a variety of ways. They may be employed as wettable powders, granules or dusts, by mixing with various inert materials, such as inorganic minerals (silicone or silicon derivatives, phyllosilicates, carbonates, sulfates, phosphates, and the like) or botanical materials (powdered corncobs, rice hulls, walnut shells, and the like). The formulations or compositions containing genetically modified bacteria may include spreader-sticker adjuvants, stabilizing agents, other pesticidal additives, or surfactants. Liquid formulations may be employed as foams, suspensions, emulsifiable concentrates, or the like. The ingredients may include Theological agents, surfactants, emulsifiers, dispersants, or polymers.

Compositions of the invention, which may include genetically modified symbiotic door bacteria expressing heterologous RNA polynucleotides, can be used for the bio-control of pathogens in an animal or other host. Such an application comprises administering to a host an effective amount of the composition which expresses from the donor sufficient heterologous RNA polynucleotides that may be transported out of the donor and taken-up by the target pathogen, thus interfering with expression of a target essential gene, and thereby controlling the pathogen and/or pathogen's disease causing effects on the host.

Compositions of the invention can be used for the control of pathogen gene expression in vivo. Such an application comprises administering to target host, such as shrimp, an effective amount of the composition which suppresses the pathogen carried by the host, reducing or eliminating the disease state in the host as well as rendering the pathogen non-transferrable, for example to a host population. Thus, regardless of the method of application, the amount of the genetically modified symbiotic door bacteria expressing heterologous RNA polynucleotides that may be applied at an effective amount to kill or suppress a pathogen, will vary depending on factors such as, for example, the specific host to be controlled, the type of pathogen, in some instances the water source to be treated, the environmental conditions, and the method, rate, and quantity of application of the composition. The concentration of the composition that is used for environmental, systemic, or foliar application will vary widely depending upon the nature of the particular formulation, means of application, environmental conditions, and degree of biocidal activity.

According to some embodiments, the heterologous asRNA polynucleotide is provided in amounts effective to reduce or suppress expression of at least one pathogen gene product. As used herein “a suppressive amount” or “an effective amount” or a “therapeutically effective amount” refers to an amount of asRNA which is sufficient to downregulate (reduce expression of) the target gene by at least 5%, 10% 20%, 30%, 40%, 50%, or more, say 60%, 70%, 80%, 90%, or even up to 100%. All ranges include the ranges in between those specifically stated.

As used herein, the term “gene” or “polynucleotide” refers to a single nucleotide or a polymer of nucleic acid residues of any length. The polynucleotide may contain deoxyribonucleotides, ribonucleotides, and/or their analogs, and may be double-stranded or single stranded. A polynucleotide can comprise modified nucleic acids (e.g., methylated), nucleic acid analogs or non-naturally occurring nucleic acids, and can be interrupted by non-nucleic acid residues. For example, a polynucleotide includes a gene, a gene fragment, cDNA, isolated DNA, mRNA, tRNA, rRNA, and isolated RNA of any sequence, recombinant polynucleotides, primers, probes, plasmids, and vectors. Included within the definition, are nucleic acid polymers that have been modified, whether naturally or by intervention.

As used herein the terms “approximately” or “about” refer to ±10%. Whenever a numerical range is indicated herein, it is meant to include any cited numeral (fractional or integral) within the indicated range. The phrases “ranging/ranges between” a first indicated number and a second indicated number and “ranging/ranges from” a first indicated number “to” a second indicated number are used herein interchangeably and are meant to include the first and second indicated numbers and all the fractional and integral numerals there between.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references, unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity, and should not be construed as an inflexible limitation on the scope of the invention. Accordingly, the description of a range should be considered to have specifically disclosed all the possible subranges as well as individual numerical values within that range. For example, description of a range, such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts. As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As used herein, “symbiotic” or “symbionts” generally refers to a bacterium that is a symbiont of a host. It may also include bacteria that persist throughout the life-cycle of a host, either internally or externally, and may further be passed horizontally to the offspring or eggs of a host. Symbionts can also include bacteria that colonize outside of host's cells and even in the tissue, lymph or secretions of the host. Endosymbionts generally refers to a subgroup of internal symbionts.

As used herein, “transbiotic” refers to the production of RNA polynucleotides inside naturally occurring or symbiotic bacterium that live within the target host organism that are designed to inhibit expression of target host or pathogen genes.

This invention utilizes routine techniques in the field of molecular biology. Basic texts disclosing the general methods of use in this invention include Green and Sambrook, 4th ed. 2012, Cold Spring Harbor Laboratory; Kriegler, Gene Transfer and Expression: A Laboratory Manual (1993); and Ausubel et al., eds., Current Protocols in Molecular Biology, 1994-current, John Wiley & Sons. Unless otherwise noted, technical terms are used according to conventional usage. Definitions of common terms in molecular biology maybe found in e.g., Benjamin Lewin, Genes IX, published by Oxford University Press, 2007 (ISBN 0763740632); Krebs, et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

To demonstrate transbiotic regulation of bacterial gene expression in a recipient pathogenic bacterium by asRNA expressed and delivered by a donor bacterium, the present inventors performed the following series of experiments. First, the present inventors used green fluorescent protein (GFP) as reporter for quantifying the expression level of a gene targeted for asRNA suppression and showed that co-cultivation of GFP-expressing bacteria (recipient) with bacteria expressing specific asRNA targeting GFP (donor) leads to a reduction in the level of GFP fluorescence in the recipient strain.

Second, the present inventors designed asRNA targeting an essential gene, dam, in the general pathogen Vibrio harveyi and demonstrated that bacteria expressing asRNA-Dam are able to suppress expression of Dam in Vibrio and alter Dam-dependent Vibrio traits, leading to suppression of Vibrio bacterial populations and pathogenic states. The Vibrio harveyi Dam gene which encodes the dam gene is involved in DNA methylation, DNA mismatch repair, regulation of DNA replication, and regulation of gene expression. Dam is also involved in regulation of the virulence pathway in many bacteria (Julio at all, 2001).

Finally, the present inventors introduced Enterobacter sp (Ag1) expressing asRNA-Dam targeting the Vibrio dam gene into C. elegans infected with V. harveyi to confirm inter-bacterial asRNA-mediated regulation of essential gene expression in a host-pathogen system resulting in substantial reductions in pathogenic bacteria populations.

### Example 1: GFP Fluorescence Level is Reduced in GFP-Expressing Bacteria Following Co-Cultivation with asRNA-GFP Expressing Donor-Bacteria Strain

The present inventors demonstrate that exemplary GFP fluorescence may be reduced through the novel transbiotic system described herein. As understood by those skilled in the art, GFP is often used as reporter for protein expression level. Furthermore, there are well characterized asRNA sequences which have been shown to suppress GFP fluorescence when GFP and asRNA-GFP, identified as SEQ ID NO. 2, are expressed in the same bacteria cell. Here, the present inventors utilized a known sequence of asRNA complimentary to the beginning of GFP coding sequence to determine if this asRNA would suppress GFP fluorescence when GFP and asRNA-GFP are expressed in different bacteria further stabilized as an asRNA loop flanked by a complimentary GC-rich dsRNA stem. As shown generally herein, the present inventors demonstrate that, this stem-loop structure allows a determination if the presence or absence of the dsRNA specific nuclease, RNase III, in recipient bacteria would impact the effectiveness of the asRNA stem-loop structure in silencing the targeted RNA-GFP. For this purpose, the present inventors used both wild type RNase III Ag1 strain and RNase III deficient HT-115 E. coli strain as recipient bacteria in these experiments.

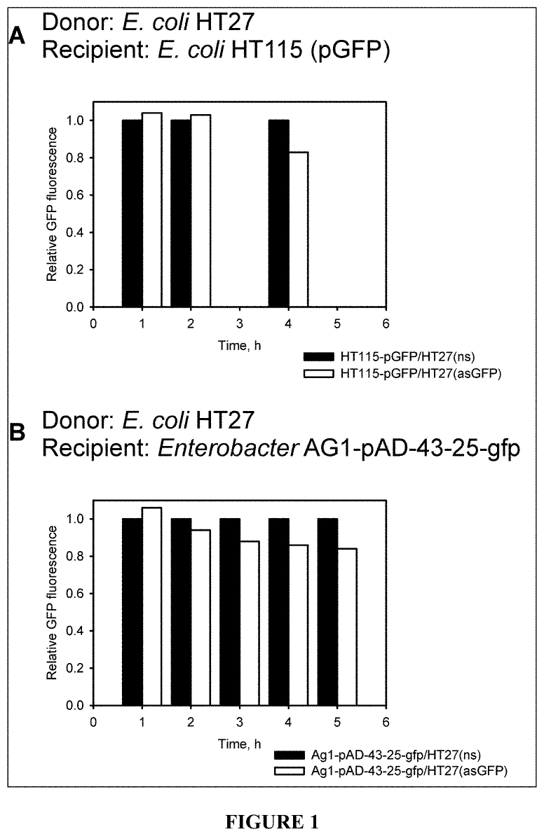

As shown in FIG. 1, to evaluate if the production of asRNA-GFP in a donor bacterium would reduce expression of GFP in acceptor bacteria, the present inventors co-cultivated donor and recipient bacteria strains together. The relative level of GFP fluorescence was measured in GFP-expressing bacteria after 4-7 h of co-cultivation of donor and recipient bacterial strains expressing either specific asRNA-GFP, or unspecific asRNA targeting COP1 gene, identified as SEQ ID NO. 4, from Aedes aegypti. Both RNase III-deficient E. coli HT-115 strain and wild type RNase III Ag1 bacteria were used to express GFP to check if presence of RNase III would affect preservation of hairpin asRNA-GFP structure in the recipient bacterium. In both Ag1 and RNase III deficient E. coli strain HT-115 the level of GFP fluorescence was reduced by ˜15% after 4 h co-cultivation with donor bacteria expressing asRNA-GFP. A negative control donor strain was E. coli HT-27 expressing an unspecific RNA (HT27 ns). The present inventors observed no effect of this strain on GFP expression in the recipient strain indicating that the reductions in GFP expression observed in the recipient strain co-cultivated with donor strains expressing asRNA-GFP was due to the delivery of asRNA-GFP to the recipient strain and subsequent partial silencing of GFP expression. (See FIG. 1).

### Example 2: Reduction of Expression of Dam Gene in Vibrio Harveyi by Specific asRNA-Dam Expressed by Enterobacter sp Ag1

As previously stated, the present invention provides a robust method for targeted suppression of essential gene expression in pathogenic bacteria by specific asRNA delivered by engineered bacteria growing in the host. As shown below, the exemplary pathogen Vibrio harveyi may be targeted for essential gene expression.

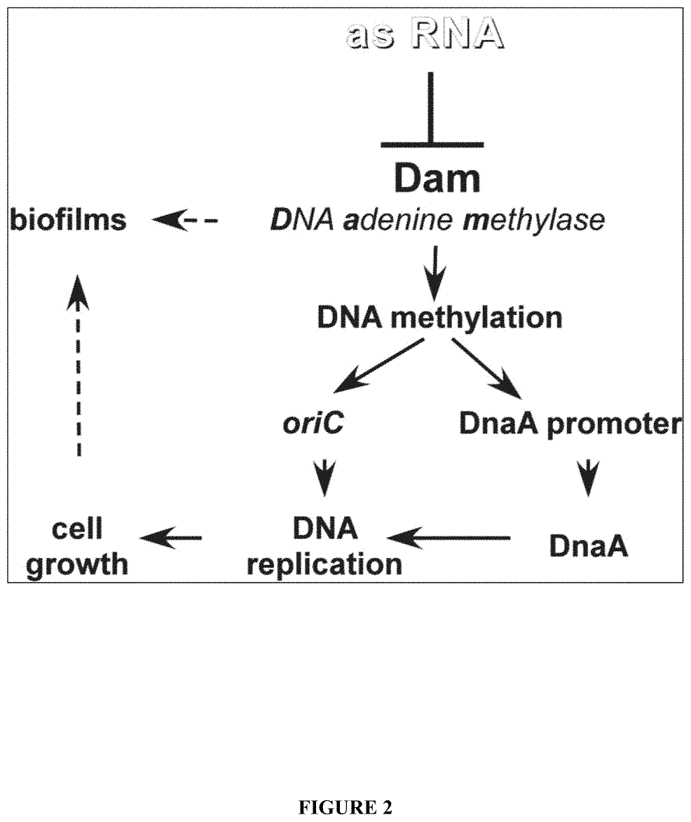

Vibrio harveyi is an opportunistic pathogen bacterium. Many Vibrio sp are common pathogens of aquaculture animals such as shrimp, oyster, prawn, lobster and many fish species. Control of Vibrio-related diseases is an important measure in aquaculture development. The Vibrio harveyi Dam (DNA adenine methyltransferase) gene encoding deoxyadenosine methylase is an exemplary target for asRNA-mediated gene silencing to suppress bacterial population growth and bacterial pathogenesis. As outline generally in FIG. 2, dam is an essential gene in Vibrio sp., and is involved in mismatch repair of DNA, regulation of replication and regulation of gene expression. Dam is also involved in regulation of virulence pathways in many bacteria.

### Example 3: Co-Cultivation of Vibrio with asDam-Expressing Ag1 Leads to Reduced Vibrio Fitness and Reduced Biofilm Formation

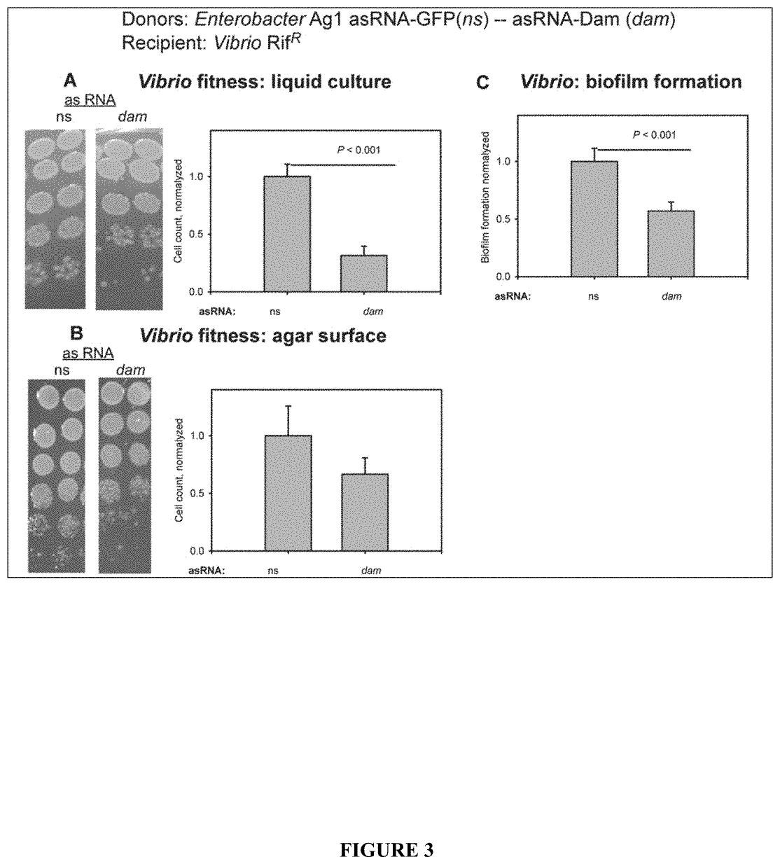

The dam (DNA adenine methyltransferase) gene plays an essential role in Vibrio DNA replication. Thus, a reduction in Dam protein levels should lead to reduced bacterial replication and, as a result, decreased cell growth. To determine whether asRNA-Dam produced in a donor bacterium could affect population growth in a recipient bacterium, the present inventors co-cultivated donor and recipient bacterium and followed population growth. The number of Vibrio cells was compared after 24 h of co-cultivation with asRNA-Dam expressing donor bacterial strains (Ag1) both in liquid culture and on agar plates. Mixed bacteria cultures were plated on LB-agar plates with 50 mg/L rifamycin in serial 10-fold dilution. As shown in FIGS. 3 A-B, since only Vibrio strain had resistance to rifamycin, this plating allowed the present inventors to discriminate between donor and recipient strains and to determine the impact of asRNA-Dam delivered to Vibrio on its growth. As shown, the present inventors observed a 3-fold reduction in Vibrio cell numbers when cultivated with donor bacterium expressing asRNA-Dam compared to controls expressing a mock asRNA when grown in liquid media and a 2-fold reduction when grown on an agar surface.

The present inventors next determined if suppression of Dam protein expression impacted the expression of pathogenesis associated traits in the recipient bacterium. Specifically, the present inventors monitored biofilm formation; a process associated with advanced pathogenesis states in bacteria. To evaluate biofilm formation in mixed culture, the present inventors co-cultivated donor and recipient bacterium for 24 h. Then biofilm formation was measured using a crystal violet staining method for the quantification of biofilm levels. It was shown by the present inventors that donor bacteria expressing asRNA-Dam reduced the formation of biofilms in Vibrio by 50% relative to controls (FIG. 3C). Since Dam is involved in regulation of biofilm formation, the decrease in biofilm formation is attributed to reduced expression of Dam protein in Vibrio in presence of bacteria expressing asRNA-Dam.

### Example 4: Overall N6-Methyladenosine (6^{m}A) DNA Methylation Decreased as Result of Co-Growth of Vibrio with Ag1-asRNA-Dam

In addition to demonstrating the decreased fitness of Vibrio cells in embodiments described above resulting from inhibition of Dam function, the present inventors further demonstrate that methylation of Vibrio DNA is also decreased. To determine if co-cultivation of Vibrio with Enterobacter Ag1-expressing asRNA-Dam had an effect on DNA methylation of Vibrio genomic DNA, dot blot experiments were performed using primary antibodies specific to 6^{m}A modified adenine.

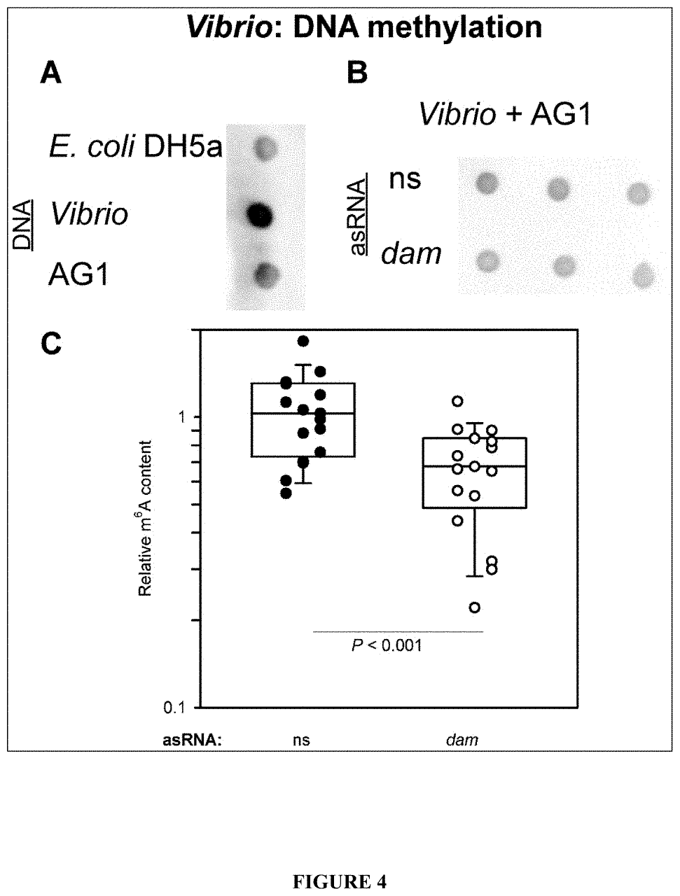

First, the present inventors analyzed the relative methylation status of genomic DNA of E. coli, V. harveyi and Enterobacter sp. The present inventors found that compared to DNA of other bacteria, V. harveyi DNA is heavily methylated (See FIG. 4A). To demonstrate the presence of asRNA-Dam expressed by Ag1 bacteria affects the level of Vibrio DNA methylation, bacterial DNA was purified from the mixed bacterial cultures (e.g., Vibrio and Ag1-asRNA-Dam; Vibrio and Ag1-asRNA-GFP) grown on agar surface and used in dot blot analysis for assessment of DNA methylation using methylation-specific antibodies as described generally herein. As shown in FIG. 4C, the present inventors observed a decrease in the intensity of immune response signal from DNA isolated co-cultivation of Vibrio and Enterobacter (donor) Ag1 expressing asRNA-Dam (See FIG. 4B). Methylation of the DNA obtained after co-growth with Ag1-asRNA-Dam was 30% lower than the signal obtained for negative non-specific control (Ag1-asRNA-GFP). Thus, the present inventors have demonstrated that after co-growth with Ag1-asRNA-Dam, the genomic DNA of Vibrio had substantially reduced methylation.

### Example 5: Decrease in the DNA Methylation Level is Due to Decrease in Methylation of Vibrio DNA

To verify that the decrease in overall level of methylation was due to specific inhibition of Dam synthesis by -asRNA-Dam, the present inventors performed methylation sensitive restriction DNA analysis of Vibrio genomic DNA using restriction endonucleases MboI and DpnI that have differential methylation sensitivity. For example, MboI cuts only dam-unmethylated DNA, and correspondingly, inhibition of Dam activity in the cells leads to a decrease in concentration of undigested fragments in the digestion mix. In contrast, DpnI cuts only dam-methylated DNA, and a decrease in DNA methylation status will lead to an accumulation of undigested DNA fragments.

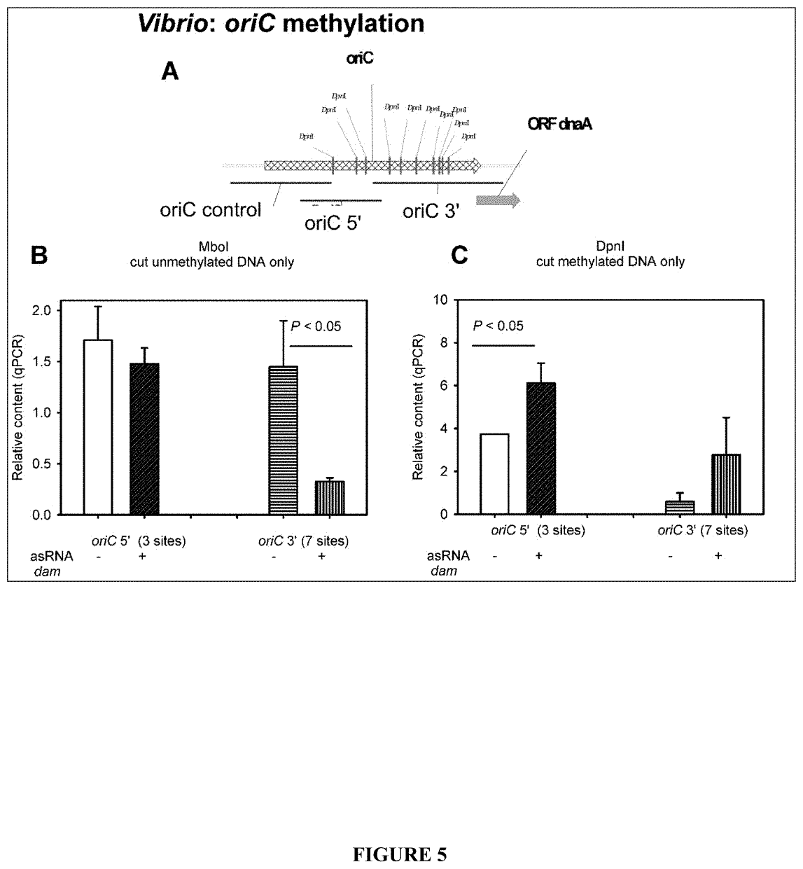

The yield of undigested DNA fragments was quantified by qPCR. Oligonucleotides specific to Vibrio oriC used in the assay are presented in Table 4 below, and the rationale for their design is shown on FIG. 5A. Briefly, for methylation sensitive restriction analysis, the present inventors choose the heavily methylated origin of replication, oriC. A region of oriC void of DpnI/MboI restriction sites was used as internal control in qPCR. Two primer sets, oriC 5′ and oriC 3′, containing 3 and 7 Dam-methylation sites respectively (DpnI/MboI), were designed to evaluate methylation status across the oriC region.

As shown in FIG. 5, the present inventors demonstrated that in the case of decreased DNA methylation, the abundance of MboI undigested fragments was decreased (5 folds, in the case of 3′ fragment (See FIG. 5 B)). Correspondingly, the abundance of DpnI fragments increased 2-4 folds (See FIG. 5 C). The results confirm that co-growth of Vibrio with Ag1-asRNA-Dam leads to inhibition of Dam activity in the Vibrio cells associated with a decrease in the DNA methylation status of oriC, leading to a reduction in Vibrio bacterial growth.

### Example 6: Co-Growth of Vibrio with Ag1-asDam Leads to Reduction of Dam mRNA

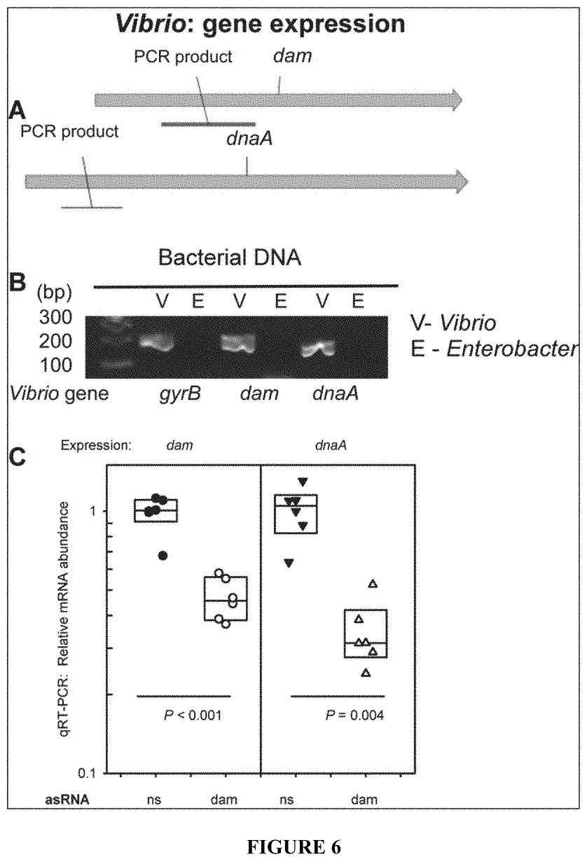

To affect the methylation status of Vibrio DNA, asRNA-Dam must enter into Vibrio cells and alter the activity and/or concentration of Dam protein. Here, the present inventors designed asRNA-Dam to overlap the start codon and potential ribosome-binding site of the Dam mRNA. Correspondingly, in one preferred embodiment, asRNA-Dam may function by preventing translation of Dam and/or by prompting mRNA degradation. To establish the potential mechanism of asRNA-Dam, the present inventors accessed the concentration of dam mRNA in total RNA. As generally demonstrated in FIG. 6, a two-fold reduction in dam mRNA was observed in Vibrio co-cultivated with Enterobacter Ag1 expressing asRNA-Dam compared to control co-cultivation with bacteria expressing non-specific asRNA-GFP. The results of qPCR experiments confirm that the level of dnaA-mRNA was 3-fold lower in Vibrio cells that were co-cultivated with Ag1 expressing asRNA-Dam than those co-cultivated with Ag1 expressing asRNA-GFP. As a result, the present inventors demonstrate that anti-sense RNA complimentary to 5′ end of dam gene expressed in the Enterobacter Ag1 is entering into Vibrio cells and inhibits the synthesis of Dam protein by destruction of dam mRNA.

### Example 7: Reduction of Expression of Dam Gene in Vibrio Harveyi by Specific asRNA Expressed by Enterobacter sp Ag1 in Host-Pathogen System

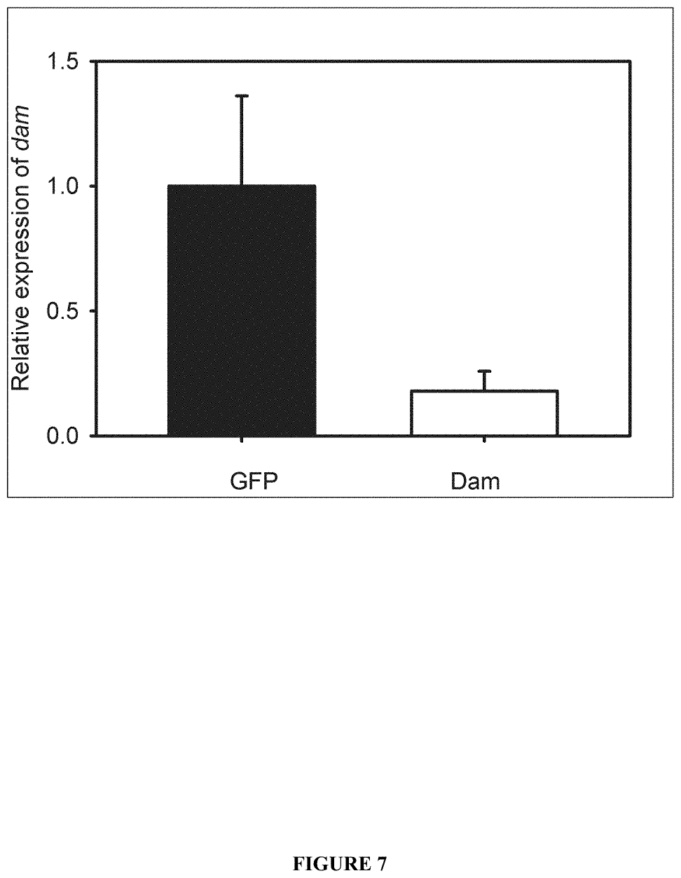

The present inventors determined that bacteria-delivered asRNA could also suppress expression of a gene of interest in targeted intestinal bacteria living in a host eukaryote. As generally shown in FIG. 7, the present inventors performed in-vivo experiments using C. elegans as an exemplary host model. C. elegans were infected with Vibrio harveyi and then fed Enterobacter Ag1 expressing asRNA-Dam or expressing asRNA-GFP for 20 h. Then total RNA was extracted from C. elegans and the level of dam mRNA was assessed by qPCR using the same primers identified in Table 3 below. Since the primers are specific only to the Vibrio dam and gyrB genes (RNA standard), the presence of C. elegans and Ag1 RNA in the total RNA didn't affect qPCR analysis. The present inventors found that expression of the Vibrio dam was 6-fold lower in C. elegans fed Enterobacter Ag1 expressing asRNA-Dam compared to C. elegans fed Ag1 bacteria expressing non-specific asRNA-GFP (See FIG. 7).

### Material and Methods

### AsRNA-Expressing Cassette Design

The paired termini (PT) RNA-stabilizing design for producing anti-sense RNA (asRNA) developed by Nakashima et al., (2006) was used for creating asRNA-expressing cassettes. 38 bp-long flanking inverted GC-reach fragments were added on both sides of the specific asRNA sequence forming a hairpin structure with the asRNA-loop at the end. EcoRV and XhoI restriction sites were added to the end of flanking inverts for convenience of cloning different asRNA sequences into cassette. RrnB terminator (terminator from rrnB E. coli gene) was placed after a 207 bp long connector sequence at the end of asRNA-expressing cassette, and the cassette was cloned into pAD-43-25 plasmid under control of the Pupp promoter using the XbaI and HindIII restriction sites (See FIG. 8).

### GFP Fluorescence Measurements:

Bacteria-donor (HT27-asGFP and HT27-COP1) and bacteria-recipient (HT115-pGFP or Ag1-pad-43-25) strains were grown separately overnight in LB (Thermo-Fisher, 12780052) with 5 mg/mL chloramphenicol, and then mixed for co-cultivation experiments in a donor/recipient ratio from 5:1 to 10:1. Mixed bacteria cultures were co-cultivated for 1-7 h, and fluorescence measurements were taken by Tecan M200 plate reader. 3 independent experiments with 8 technical replicates for each experiment were analyzed for each treatment. GFP fluorescence in pAD-43-25-transformed bacteria was measured using excitation at 485 nm and emission at 528 nm, GFP uv-induced fluorescence in HT115-pGFP cells was measured at an excitation wavelength of 400 nm and emission wavelength of 520 nm.

### Cell Count Assay:

Vibrio fitness before and after treatments was determined by cell counting assays:

### Co-Cultivation in Liquid Culture Experiments:

Vibrio-Rif^{R} (asRNA recipient strain) and Ag1-asRNA-Dam1 or Ag1-asRNA-GFP1 (asRNA donor strains) were grown overnight in LBS (10 g/L Bacto-Tryptone, 5 g/L yeast extract, 20 g/L NaCl, 50 mM Tris-HCl pH 7.5) medium, diluted to OD (600 nm) of 0.2-0.4 and were mixed in a ratio of donor/recipient of 5/1. Mixed cultures were grown on 28° C. for 24 h and then plated on agar-LBS plates with 50 mg/L rifamycin as 5 μL of 10-fold serial dilutions. Bacteria were grown overnight on 28° C. before cell count. 3 independent experiments were performed for this analysis.

### Co-Growth on Agar Surface:

5 μL of overnight donor strain and 5 μl of recipient strain were simultaneously dropped on LBS agar plate and grown for 24 h at 28° C. Then the mixed bacterial spot was cut out of the agar layer, dissolved in PBS and 5 μL plated on agar-LBS plates with 50 mg/L rifamycin in 10-fold serial dilutions. 8 independent experiments were used for this analysis.

### Biofilm Formation Assay

Vibrio harveyi and Ag1-pAD-pt-Dam1 or Ag1-pAD-pt-GFP1 were grown overnight in LBS medium, diluted to OD₆₀₀ 0.2-0.4 and mixed in a ratio of donor/recipient at 5/1. Then 100 μl mixed culture were added into wells in 96 well plates (3 independent experiments with 8 technical replicates in each experiment were analyzed for each treatment) and incubated without shaking on 28° C. for 24 h. After incubation, the bacterial biofilm was stained by crystal violet according to the protocol described by O'Toole (O'Toole, 2011) and absorbance was measured on Tecan plate reader at 550 nm.

### N6-Methyladenosine (6^{m}A) Dot Blot Analysis

Herr, 6^{m}A abundance in bacterial DNA was measured by dot blot assay using mouse antibody raised against DNA with N6-methyladenine (6^{m}A). Bacterial DNA was purified using Omega bacterial DNA Purification Kit. DNA was then diluted to a concentration of 100 ng/μl with 8 M urea. DNA samples were denatured by heating at 95° C. for 3 min. Samples were chilled on ice immediately after denaturation to prevent the re-formation of secondary structure. Duplicates of 2 μl were applied to an Amersham Hybond-N+ membrane (GE Healthcare). UV crosslinking of DNA to the membrane was performed by running the auto-crosslink program twice using a Stratalinker 2400. All procedures were performed at room temperature. After PBST (137 mM NaCl, 12 mM Phosphate, 2.7 mM KCl, pH 7.4 and 0.1% Tween 20) wash, primary mouse anti-6^{m} A antibody (Synaptic Systems; 212B11) at 1:1000 dilution was applied for 2 h incubation at RT. After 3 washes in excess of PBST for 30 min, the membrane was incubated in HRP-conjugated anti-mouse IgG secondary antibody (Thermo), then washed again 3 times in excess of PBST for 30 min. Finally, the antibody signal was visualized using SuperSignal West Dura Extended Duration Substrate (Thermo Scientific; 34075). To confirm equal DNA loading, the same membrane was stained with Sybr Green fluorescent dye and DNA was visualized using Gel Doc Easy imager (BioRad). Quantified 6^{m}A levels were normalized to the amount of DNA loaded. Dot blot analysis was repeated using 3-8 independent biological samples and 2-3 technical repeats. Signals from the dot blot images were quantified by ImageJ and were the subject of the statistical analysis. Results were plotted using SigmaPlot.

qRT-PCR.

Relative gene expression in Vibrio cells was measured by quantitative real-time PCR (qRT-PCR). Total RNAs were isolated using an Omega E.Z.N.A Bacterial RNA kit. Real-time PCR amplification was performed by using an Mx3000P QPCR system (Agilent technologies). A Power SYBR® Green RNA to C_{T}™ 1-Step Kit (Applied Biosystems) was used to perform one step RT-PCR. Oligonucleotides concentration and cycling conditions used were according manufacturer recommendations. Gene specific primers are listed in Table 2. 25 ng of total bacterial RNA was used in each reaction. Relative expression levels of the specific transcripts were calculated using the gyrB mRNA expression level as the internal reference for normalization.

### Restriction Digestion Assays

To determine methylation status of Vibrio origin oriC, genomic DNA was cleaved independently with specific restriction endonucleases in order to decipher the presence or the absence of 6^{m}A. Bacterial genomic DNA was purified using Omega E.Z.N.A. Bacterial DNA Kit. Separate digestions of 500 ng of DNA using 5 U by each restriction enzyme (MboI, cut unmethylated DNA only and DpnI, cut methylated DNA only (Thermo)) were performed for 3 h at 37° C. To determine relative abundance of DNA fragments that remained intact following the restriction enzyme digestion, qPCR was performed using the PowerUp SYBR Green Master Mix kit (Applied Biosystems). Gene specific primers are listed in Table 2 below. Relative levels of the specific DNA fragments were determined using the level of methylation-free DNA fragment as the internal reference for normalization.

Caenorhabditis elegans Assay.

C. elegans were used as a model animal for studying of effect of heterogeneously expressed asRNA on level of gene expression in Vibrio in host-pathogen system. C. elegans N2 strain, was grown on solid standard nematode growth medium (NGM) plates at 25° C. and fed E. coli OP50. The worms were then synchronized in the dauer stage by plating to empty NGM plates. Synchronous dauer cultures were then transferred to NGM plates with Vibrio harveyi for 48 h. Then worms were washed 3 times with M9 buffer, re-suspended and divided on two halves, one was plated to Ag1-asDam NGM plates, and second to Ag1-asGFP plates. After 20 h of feeding by Ag1 bacteria, nematodes were washed 3 times with M9, double volume of RNAprotect Bacteria reagent (Qiagen) was added to buffer, and then worms were disrupted by intensive vortexing with metal grids to released intestinal bacteria and used for total RNA extraction. Relative dam gene expression in Vibrio cells was measured by quantitative real-time PCR (qRT-PCR) as described above.

### Data Analyses.

Averages and standard errors of the mean (SEM) were calculated from at least three independent experiments. All other data were analyzed by Anova test with SigmaPlot. Significance of differences between experimental groups was accepted at a P value of <0.05.

### REFERENCES

The following references are hereby incorporated in their entirety by reference:
[1] Yadav, M. K., Y. Y. Go, S. W. Chae & J. J. Song, (2015) The Small Molecule DAM Inhibitor, Pyrimidinedione, Disrupts Streptococcus pneumoniae Biofilm Growth In Vitro. PLoS One 10: e0139238.
[2] Berenstein, D., K. Olesen, C. Speck & O. Skovgaard, (2002) Genetic organization of the Vibrio harveyi DnaA gene region and analysis of the function of the V. harveyi DnaA protein in Escherichia coli. J Bacteriol 184: 2533-2538.
[3] Collier, J., H. H. McAdams & L. Shapiro, (2007) A DNA methylation ratchet governs progression through a bacterial cell cycle. Proc Natl Acad Sci USA 104: 17111-17116.
[4] Hoynes-O'Connor, A 3. asRNA-Dam alignment to dam mRNA. & T. S. Moon, (2016) Development of Design Rules for Reliable Antisense RNA Behavior in E. coli. ACS Synth Biol 5: 1441-1454.
[5] Julio, S. M., D. M. Heithoff, D. Provenzano, K. E. Klose, R. L. Sinsheimer, D. A. Low & M. J. Mahan, (2001) DNA Adenine Methylase Is Essential for Viability and Plays a Role in the Pathogenesis of Yersinia pseudotuberculosis and Vibrio cholerae. Infection and immunity 69: 7610-7615.
[6] Nakashima, N., T. Tamura & L. Good, (2006) Paired termini stabilize antisense RNAs and enhance conditional gene silencing in Escherichia coli. Nucleic Acids Res 34: e138.
[7] O'Toole, G. A., (2011) Microtiter dish biofilm formation assay. J Vis Exp.
[8] Val, M. E., S. P. Kennedy, A. J. Soler-Bistue, V. Barbe, C. Bouchier, M. Ducos-Galand, O.
[9] Skovgaard & D. Mazel, (2014) Fuse or die: how to survive the loss of Dam in Vibrio cholerae. Mol Microbiol 91: 665-678.

### Tables

TABLE 1
Bacterial strains.

| Strain | | |
|---|---|---|
| name | Description | Source |
| Vibrio | ATCC 14126 | ATCC |
| harveyi | | |
| Vibrio | Spontaneous Rif^{R} isolate of V. harveyi | This work |
| | ATCC 14126 | |
| Ac1 | Enterobacter sp | Obtained from |
| | | NMSU, Xu lab, |
| | | Aedes gambiae |
| | | midgut isolate |
| Ag1- | AG1 (pAD Dam) Ap^{R}, asRNA to Vibrio | Plasmid made by |
| asDam | dam under Pupp cloned into pAD43-25 | GENESCRIPT |
| | | transformed into |
| | | Ag1 |
| Ag1-asGFP | AG1 (pAD GFP) Ap^{R}, asRNA to gfp | Plasmid made by |
| | under Pupp cloned into pAD43-25 | GENESCRIPT |
| | | transformed into |
| | | Ag1 |
| HT-115 | E. coli RNase III mutant | |
| Ht27 | E. coli RNase III mutant | |
| Ag1-pAD- | Ag1 (pAD43-25) GFP expressing strain, | Present invention |
| 43-25 | Ap^{R} | |
| Ht115- | E. coli Ht115 (pGFPuv) Ap^{R} | Present invention |
| pGFPuv | | |
| Ht27- | E. coli Ht27 (pAD GFP) Ap^{R}, asRNA to | Present invention |
| asGFP | gfp under Pupp cloned into pAD43-25 | |
| Ht27-COP | E. coli Ht27 (pAD GFP) Ap^{R}, asRNA to | Present invention |
| | COP under Pupp cloned into pAD43-25 | |

TABLE 2
Gene specific primers.

| RNA part | RNA sequence |
|---|---|
| Paired | CAGGAGGAAUUAACCAUGCAGUGGUGGUGGUGGUGGUG |
| termini 1 | |
| Paired | CACCACCACCACCACCACUGCAUGGUUAAUUCCUCCUG |
| termini 2 | |
| asRNA-GFP | UAAUUCAACAAGAAUUGGGACAACUCCAGUGAAAAGU |
| | UCUUCUCCUUUACUCAU |
| asRNA-Dam | CUUUUUCAUCUACUGCUCUAUCUAUCGACCAAAAAUUAAG |
| | GCUGCGGAAUGUAACAUAU |

TABLE 3
Oligonucleotides used in qRT-PCR.

| Oligo name | DNA sequence |
|---|---|
| Vhv gyrB-for | TTA GGT GCT CAA CGA ACG CT |
| Vhv gyrB-rev | TAC ACA GAT CCG CTT CTG GC |
| Vhy dnaA-for | CTT TCT TCG CAC CTA ACC GC |
| Vhy dnaA rev | TGA AGA CTC TGC CGC AAC AT |
| Vhy dam-for | GGC GGA TAT TAA CCC CGA CC |
| Vhy dam-rev | GCC ATT AAA GCC AAA GCG GT |

TABLE 4
Oligonucleotides used in qPCR analysis.

| Oligo name | DNA sequence |
|---|---|
| OriC_Cntrl | ACC TTG TAG CTT ATC TTG CTT CAC |
| for | |
| OriC_Cntrl | TTA CTC TGA GGT CTA GGT TAT TGC |
| rev | |
| OriC_5′ for | TGG AAA TAC TAC TGT CAG TTA GCT C |
| OriC_5′ rev | CTG TGT ATG AAT TTT CAT TCA TCG G |
| OriC_3′ for | TGA AAA TTC ATA CAC AGA GTT ATC ACC |
| OriC_3′ rev | TGT AGC TGG TAG CTC TTC TTG |

TABLE 5
Exemplary donor enteric bacteria.

Acidimicrobiia
Actinobacteria
Alphaproteobacteria
Anaerolineae
Bacilli
Bacteroidia
Betaproteobacteria
Clostridia
Deltaproteobacteria
Epsilonproteobacteria
Flavobacteriia
Fusobacteria
Gammaproteobacteria
Mollicutes
Opitutae
Oscillatoriophycideae
Phycisphaerae
Planctomycetia
Rubrobacteria
Sphingobacteriia
Synechococcophycideae
Thermomicrobia
Verrucomicrobiae

TABLE 6
Gene targets related to MDR in pathogenic bacteria.

Aminocoumarins
Aminocoumarin-resistant DNA topoisomerases
Aminocoumarin-resistant GyrB, ParE, ParY
Aminoglycosides
Aminoglycoside acetyltransferases
AAC(1), AAC(2′), AAC(3), AAC(6′)
Aminoglycosidc nucicotidyltransferases
ANT(2″), ANT(3″), ANT(4′), ANT(6), ANT(9)
Aminoglycoside phosphotransferases
APH(2″), APH(3″), APH(3′), APH(4), APH(6), APH(7″), APH(9)
16S rRNA methyltransferases
ArmA, RmtA, RmtB, RmtC, Sgm
β-Lactams
Class A β-lactamases
AER, BLA1, CTX-M, KPC, SHV, TEM, etc.
Class B (metallo-)β-lactamases
BlaB, CcrA, IMP, NDM, VIM, etc.
Class C β-lactamases
ACT, AmpC, CMY, LAT, PDC, etc.
Class D β-lactamases
OXA β-lactamaseb
mecA (methicillin-resistant PBP2)
Mutant porin proteins conferring antibiotic resistance
Antibiotic-resistant Omp36, OmpF, PIB (por)
Genes modulating β-lactam resistance
bla (blaI, blaR1) and mec (mecI, mecR1) operons
Chloramphenicol
Chloramphenicol acetyltransferase (CAT)
Chloramphenicol phosphotransferase
Ethambutol
Ethambutol-resistant arabinosyltransferase (EmbB)
Mupirocin
Mupirocin-resistant isoleucyl-tRNA synthetases
MupA, MupB
Peptide antibiotics
Integral membrane protein MprF
Phenicol
Cfr 23S rRNA methyltransferase
Rifampin
Rifampin ADP-ribosyltransfcrase (Arr)
Rifampin glycosyltransferase
Rifampin monooxygenase
Rifampin phosphotransferase
Rifampin resistance RNA polymerase-binding proteins
DuaA, RbpA
Rifampin-resistant beta-subunit of RNA polymerase (RpoB)
Streptogramins
Cfr 23S rRNA methyltransferase
Erm 23S rRNA methyltransferases
ErmA, ErmB, Erm(31), etc.
Streptogramin resistance ATP-binding cassette (ABC) efflux pumps
Lsa, MsrA, Vga, VgaB
Streptogramin Vgb lyase
Vat acetyltransferase
Fluoroquinolones
Fluoroquinolone acetyltransferase
Fluoroquinolone-resistant DNA topoisomerases
Fluoroquinolone-resistant GyrA, GyrB, ParC
Quinolone resistance protein (Qnr)
Fosfomycin
Fosfomycin phosphotransferases
FomA, FomB, FosC
Fosfomycin thiol transferases
FosA, FosB, FosX
Glycopeptides
VanA, VanB, VanD, VanR, VanS, etc.
Lincosamides
Cfr 23S rRNA methyltransferasc
Erm 23S rRNA methyltransferases
ErmA, ErmB, Erm(31), etc.
Lincosamide nucleotidyltransferase (Lin)
Linezolid
Cfr 23S rRNA methyltransferase
Macrolides
Cfr 23S rRNA methyltransferase
Erm 23S rRNA methyltransferases
ErmA, ErmR, Erm(31), etc.
Macrolide esterases
EreA, EreB
Macrolide glycosyltransferases
GimA, Mgt, Ole
Macrolide phosphotransferases (MPH)
MPH(2′)-I, MPH(2′)-II
Macrolide resistance efflux pumps
MefA, MefE, Mel
Streptothricin
Streptothricin acetyltransferase (sat)
Sulfonamides
Sulfonamide-resistant dihydropteroate synthases
Sul1, Sul2, Sul3, sulfonamide-resistant FolP
Tetracyclines
Mutant porin PIB (por) with reduced permeability
Tetracycline inactivation enzyme TetX
Tetracycline resistance major facilitator superfamily (MFS) efflux
pumps
TetA, TetB, TetC, Tet30, Tet31, etc.
Tetracycline resistance ribosomal protection proteins
TetM, TetO, TetQ, Tet32, Tet36, etc.
Efflux pumps conferring antibiotic resistance
ABC antibiotic efflux pump
MacAB-TolC, MsbA, MsrA,VgaB, etc.
MFS antibiotic cfflux pump
EmrD, EmrAB-TolC, NorB, GepA, etc.
Multidrug and toxic compound extrusion (MATE) transporter
MepA
Resistance-nodulation-cell division (RND) efflux pump
AdeABC, AcrD, MexAB-OprM, mtrCDE, etc.
Small multidrug resistance (SMR) antibiotic efflux pump
EmrE
Genes modulating antibiotic efflux
adeR, acrR, baeSR, mexR, phoPQ, mtrR, etc.
Additional MDR gene targets
dedA
RH201207_02818
wabN
galE
wzabc
arnBCADTE
lpp
surA
RH201207_00408
RH201207_00413
rnhA
galU
fabR
RH201207_00757
Pgi Glucose-6-phosphate isomerase,
bamB
tolQRAB
Pal
dedD
sdhD
hupA
yhcB
RH201207_03912
gspA/rfbD
flcpA F
degP
RH201207_05041/42
tagA
cysC
ydgA
RH201207_01572
cpxR
fre
mrcB
miaA
envC

TABLE 7
Examples of animal pathogens that may be targeted
with the present inventive technology.

Acinetobacter baumannii
Acinetobacter lwoffii
Acinetobacter spp. (incl. MDR)
Actinomycetes
Adenovirus
Aeromonas spp.
Alcaligenes faecalis
Alcaligenes spp./Achromobacter
spp.
Alcaligenes xylosoxidans (incl.
ESBL/MRGN)
Arbovirus
Ascaris lumbricoides
Aspergillus spp.
Astrovirus
Bacillus anthracis
Bacillus cereus
Bacillus subtilis
Bacteriodes fragilis
Bartonella quintana
Blastocystis hominis
Bordetella pertussis
Borrelia burgdorferi
Borrelia duttoni
Borrelia recurrentis
Brevundimonas diminuki
Brevundimonas vesicularis
Brucella spp.
Burkholderia cepacia (incl. MDR)
Burkholderia mallei
Burkholderia pseudomallei
Campylobacter jejuni/coli
Candida albicans
Candida auris
Candida krusei
Candida parapsilosis
Chikungunya virus (CHTK V)
Chlamydia pneumoniae
Chlamydia psittaci
Chlamydia trachomatis
Citrobacter spp.
Clostridium botulinum
Clostridium difficile
Clostridium perfringens
Clostridium tetani
Coronavirus (incl. SARS- and
MERS-CoV)
Corynebacterium diphtheriae
Corynebacterium
pseudotuberculosis
Corynebacterium spp.
Corynebacterium ulcerans
Coxiella burnetii
Coxsackievirus
Crimean-Congo haemorrhagic fever
virus
Cryptococcus neoformans
Cryptosporidium hominis
Cryptosporidium parvum
Cyclospora cayetanensis
Cytomegalovirus—CMV
Dengue virus
Dientamoeba fragilis
Ebola virus
Echinococcus spp.
Echovirus
Entamoeba dispar
Entamoeba histolytica
Enterobacter aerogenes
Enterobacter cloacae (incl.
ESBL/MRG19
Enterobius vermicularis
Enterococcus faecalis (incl. VRE)
Eriterucuccus fuecium (Incl. VRE)
Enterococcus hirae
Epidermophyton spp.
Epstein-Barr virus—EBV
Escherichia coli (incl. EHEC, EPEC,
ETEC, EIEC, EAEC, ESBL/MRGN,
DAEC)
Filarial worms
Foot-and-mouth disease
virus (FMDV)
Francisella tularensis
Giardia lamblia
Haemophilus influenzae
Hantavirus
Helicobacter pylori
Helminths (Worms)
Hepatitis A virus—HAV
Hepatitis B virus—HBV
Hepatitis C virus—HCV
Hepatitis D virus
Hepatitis E virus
Herpes simplex virus—HSV
Histoplasma capsulatum
Human enterovirus 71
Human herpesvirus 6 (HHV-6)
Human herpesvirus 7 (HHV-7)
Human herpesvirus 8 (HHV-8)
Human immunodeficiency virus—
HIV
Human metapneumovirus
Human papillomavirus
Hymenolepsis nana
Influenza virus (incl. A(H1N1),
A(H1N1)pdm09, A(H3N2),
A(H5N1), A(H5N5), A(H5N6),
A(H5N8), A(H7N9), A(H10N8))
Klebsiella granulomatis
Klebsiella oxytoca (incl.
ESBL/MRGN)
Klebsiella pneumoniae MDR (incl.
ESBL/MRGN)
Lassa virus
Leclercia adecarboxylata
Legionella pneumophila
Leishmania spp.
Leptospira interrogans
Leuconostoc pseudomesenteroides
Listeria monocytogenes
Marburg virus
Measles virus
Micrococcus luteus
Microsporum spp.
Molluscipoxvirus
Morganella spp.
Mumps virus
Mycobacterium chimaera Myco
Mycobacterium leprae Myco
Mycobacterium tuberculosis (incl.
MDR) Tuberculocidal
Mycoplasma genitalium
Mycoplasma pneumoniae
Naegleria fowleri
Neisseria meningitidis
Neisseria gonorrhoeae
Norovirus
Opisthorchis viverrini
Orientia tsutsugamushi
Pantoea agglomerans
Paracoccus yeei
Parainfluenza virus
Parvovirus
Pediculus humanus capitis
Pediculus humanus corporis
Plasmodium spp.
Pneumocystis jiroveci
Poliovirus
Polyomavirus
Prevotella spp.
Prions
Propionibacterium species
Proteus mirabilis (incl.
ESBL/MRGN)
Proteus vulgaris
Providencia rettgeri
Providencia stuarrif
Pseudomonas aeruginosa
Pseudomonas spp.
Rabies virus
Ralstonia spp.
Respiratory syncytial virus—RSV
Rhinovirus
Rickettsia prowazekii
Rickettsia typhi
Roseomonas gilardii
Rotavirus
Rubella virus
Schistosoma mansoni
Salmonella enteritidis
Salmonella paratyphi
Salmonella spp.
Salmonella typhi
Salmonella typhimurium
Sarcoptes scabiei (Itch mite)
Sapovirus
Serratia marcescens (incl.
ESBL/MRGN)
Shigella sonnei
Sphingomonas species
Staphylococcus aureus (incl. MRSA,
VRSA)
Staphylococcus capitis
Staphylococcus epidermidis (incl.
MRSE)
Staphylococcus haemolyticus
Staphylococcus hominis
Staphylococcus lugdunensis
Staphylococcus pasteuri
Staphylococcus saprophyticzts
Stenotrophomonas maltophilia
Streptococcus pneumoniae
Streptococcus pyogenes (incl.
PRSP)
Streptococcus spp.
Strongyloides stercoralis
Taenia solium
TBE virus
Toxoplasma gondii
Treponerna pallidum
Trichinella spiralis
Trichomonas vaginalis.
Trichophyton spp.
Trichosporon spp.
Trichuris trichiura
Trypanosoma brucei gambiense
Trypanosoma brucei rhodesiense
Trypanosoma cruzi
Usutu virus
Vaccinia virus
Varicella zoster virus
Variola virus
Vibrio cholerae
West Nile virus (WNV)
Yellow fever virus
Yersinia enterocolitica
Yersinia pestis
Yersinia pseudotuberculosis
Zika virus

SEQUENCE LISTINGS
(asRNA-Dam)
SEQ ID NO. 1
CUUUUUCAUCUACUGCUCUAUCUAUCGACCAAAAAUUAAGGCUGCGGAAU
GUAACAUAU
(asRNA-GFP)
SEQ ID NO. 2
UAAUUCAACAAGAAUUGGGACAACUCCAGUGAAAAGUUCUUCUCCUUUAC
UCAU
(Vibrio dam gene)
SEQ ID NO. 3
ATGAAAAAGCAACGAGCCTTTCTTAAGTGGGCAGGAGGCAAATACGGTCT
GGTTGAAGACATCCAACGTCATTTACCACCGGCTCGAAAGCTAGTTGAAC
CCTTTGTTGGTGCTGGCTCGGTTTTTCTAAATACCGACTATGACCACTAT
CTACTGGCGGATATTAACCCCGACCTGATTAATCTCTATAACTTACTAAA
AGAGCGTCCTGAAGAGTACATCTCAGAAGCGAAGCGCTGGTTTGTTGCAG
AGAACAATCGCAAAGAAGCGTACTTGAATATTCGCGCCGAGTTTAATAAA
ACGGATGACGTGATGTACCGCTCGTTGGCGTTCCTATACATGAACCGCTT
TGGCTTTAATGGCTTATGTCGTTATAACAAAAAAGGCGGCTTTAATGTCC
CGTTTGGTTCTTACAAAAAGCCTTATTTCCCAGAAGCGGAGCTAGAATTC
TTTGCTGAAAAAGCCAAGAAAGCGACGTTCGTATGTGAAGGTTACCCAGA
AACGTTCAGTCGAGCGCGTAAAGGCAGCGTGGTTTATTGCGATCCACCGT
ACGCACCGTTGTCGAACACGGCGAACTTTACCTCTTATGCTGGCAACGGC
TTTACGCTGGATGATCAAGCTGCATTGGCTGATATTGCAGAGAAAGCCGC
AACTGAACGTGGTATCCCTGTTCTGATCTCAAACCATGACACGACATTAA
CGCGTCGCCTTTATCATGGTGCGGAGCTTAATGTCGTAAAAGTGAAGCGA
ACCATCAGTCGTAATGGCAGTGGTCGTAATAAAGTTGACGAGTTGCTGGC
GCTATTTCGTGCACCTGACGCGGACAAATCTGACTCTTAA
(RNA-COP1)
SEQ ID NO. 4
CCCTTCACAAACCTGGAGAAAACGTCCGTGCTGCAGGAAACGCGGATGTT
TAACGAGACCCCGGTCAATGCCCGCAAGTGTACCCACATCCTGACGAAGA
TTCTGTATTTGATCAATCAGGGAGAACAACTGGGTTCCAGAGAGGCCACC
GAATGTTTC

1-41. (canceled)

42. A method of controlling gene expression in pathogenic bacteria comprising the steps of:
generating a genetically modified donor bacteria configured to express a heterologous asRNA polynucleotide directed to an essential gene of a bacterial pathogen;
introducing said genetically modified donor bacteria to a target host that is infected with said bacterial pathogen, or is susceptible to infection by said bacterial pathogen;
expressing said heterologous asRNA polynucleotide directed to an essential gene of said bacterial pathogen;
transporting said heterologous asRNA polynucleotide directed to an essential gene of said bacterial pathogen out of said genetically modified donor bacteria;
introducing said heterologous asRNA polynucleotide directed to an essential gene of said bacterial pathogen wherein said bacterial pathogen takes up said asRNA polynucleotide; and
inhibiting expression of said essential gene of bacterial pathogen through the action of said heterologous asRNA polynucleotide hybridizing with the mRNA of said essential gene of a bacterial pathogen.

43. The method of claim 42, wherein said a target host is a shrimp.

44. The method of claim 43, wherein said genetically modified donor bacteria comprises a genetically modified donor bacteria that is symbiotic with said shrimp.

45. The method of claim 44, wherein said bacterial pathogen is a species of Vibrio bacteria.

46. The method of claim 45, wherein said Vibrio species is Vibrio Harveyi.

47. The method of claim 46, wherein said essential gene of a bacterial pathogen comprises DNA adenine methylase (Dam), identified as SEQ ID NO. 3, or a homolog thereof.

48. The method of claim 47, wherein said heterologous asRNA polynucleotide comprises a heterologous asRNA polynucleotide identified as SEQ ID NO. 1, or a homolog thereof.

49. The method of claim 43, wherein said genetically modified donor bacteria is a genetically modified probiotic-like donor bacteria.

50. The method of claim 48, wherein said genetically modified probiotic-like donor bacteria comprises Bacillus subtilis.

51. The method of claim 42, wherein said genetically modified donor bacteria comprises an RNaseIII deficient genetically modified donor bacteria.

52. The method of claim 51, wherein said genetically modified donor bacteria that are symbiotic with said shrimp is selected from the group consisting of: Enterobacter, and E. coli.

53. A method of controlling bacterial biofilm formation comprising the steps:
generating a genetically modified donor bacteria configured to express a heterologous asRNA polynucleotide directed to an essential gene that contributes to biofilm formation by a bacterial pathogen;
introducing said genetically modified donor bacteria to a target host that is infected with said bacterial pathogen, or is susceptible to infection by said bacterial pathogen;
expressing said heterologous asRNA polynucleotide directed to an essential gene that contributes to biofilm formation by said bacterial pathogen;
transporting said heterologous asRNA polynucleotide directed to an essential gene that contributes to biofilm formation by said bacterial pathogen out of said genetically modified donor bacteria;
introducing said heterologous asRNA polynucleotide directed to an essential gene that contributes to biofilm formation by said bacterial pathogen wherein said bacterial pathogen takes up said asRNA polynucleotide; and
inhibiting expression of said essential gene that contributes to biofilm formation by a bacterial pathogen through the action of said heterologous asRNA polynucleotide hybridizing with the complementary mRNA of said essential gene of a bacterial pathogen.

54. The method of claim 53, wherein said target host is a shrimp.

55. The method of claim 54, wherein said genetically modified donor bacteria comprises a genetically modified donor bacteria that is symbiotic with said shrimp.

56. The method of claim 55, wherein said bacterial pathogen is a species of Vibrio bacteria.

57. The method of claim 56, wherein said Vibrio species is Vibrio Harveyi.

58. The method of claim 57, wherein said essential gene of a bacterial pathogen comprises DNA adenine methylase (Dam), identified as SEQ ID NO. 3, or a homolog thereof.

59. The method of claim 58, wherein said heterologous asRNA polynucleotide comprises a heterologous asRNA polynucleotide identified as SEQ ID NO. 1, or a homolog thereof.

60. The method of claim 53, wherein said genetically modified donor bacteria is a genetically modified probiotic-like donor bacteria.

61. The method of claim 60, wherein said genetically modified probiotic-like donor bacteria comprises Bacillus subtilis.

62. The method of claim 42, wherein said genetically modified donor bacteria comprises an RNaseIII deficient genetically modified donor bacteria.

63. The method of claim 59, wherein said genetically modified donor bacteria that is symbiotic with said shrimp is selected from the group consisting of: Enterobacter, and E. coli.

64. A method of treating a Vibrio infection in an organism comprising the steps of:
generating a genetically modified donor bacteria configured to express a heterologous asRNA polynucleotide that is complementary to the mRNA of DNA adenine methylase (Dam) of a Vibrio bacterial pathogen;
introducing said genetically modified donor bacteria to a target host that is infected with said Vibrio bacterial pathogen, or is susceptible to infection by said Vibrio bacterial pathogen;
expressing said heterologous asRNA polynucleotide that is complementary to said mRNA of DNA adenine methylase (Dam) of said Vibrio bacterial pathogen;
transporting said heterologous asRNA polynucleotide that is complementary to the mRNA of DNA adenine methylase (Dam) of said Vibrio bacterial pathogen out of said genetically modified donor bacteria;
introducing said heterologous asRNA polynucleotide that is complementary to the mRNA of DNA adenine methylase (Dam) of said Vibrio bacterial pathogen wherein said bacterial pathogen takes up said asRNA polynucleotide; and
inhibiting expression of said essential gene of bacterial pathogen through the action of said asRNA hybridizing with the complementary mRNA of said essential gene of said Vibrio bacterial pathogen.

65. The method of claim 64, wherein said a target host is a shrimp.

66. The method of claim 65, wherein said genetically modified donor bacteria comprises a genetically modified donor bacteria that is symbiotic with said shrimp.

67. The method of claim 64, wherein said Vibrio species is Vibrio Harveyi.

68. The method of claim 67, wherein said DNA adenine methylase (Dam), is identified as SEQ ID NO. 3, or a homolog thereof.

69. The method of claim 64, wherein said heterologous asRNA polynucleotide that is complementary to the mRNA of DNA adenine methylase (Dam) of a Vibrio bacterial pathogen comprises a heterologous asRNA polynucleotide identified as SEQ ID NO. 1, or a homolog thereof.

70. The method of claim 69, wherein said genetically modified donor bacteria is a genetically modified probiotic-like donor bacteria.

71. The method of claim 70, wherein genetically modified probiotic-like donor bacteria comprises Bacillus subtilis.

72. The method of claim 64, wherein said genetically modified donor bacteria comprises an RNaseIII deficient genetically modified donor bacteria.

73. The method of claim 66, wherein said genetically modified donor bacteria that are symbiotic with said shrimp is selected from the group consisting of: Enterobacter, and E. coli.
